(19) United States
(12) Patent Application Publication
(10) Pub. No.: US 2007/0174926 A1
(43) Pub. Date: Jul. 26, 2007

(54) Sperm factor oscillogenin

(76) Inventor: Rafael A. Fissore, Amherst, MA (US)

Correspondence Address: Merchant & Gould PC, P.O. Box 2903, Minneapolis, MN, 55402-0903, US

(21) Appl. No.: 10/344,723

(22) Filed: Feb. 14, 2003

Related U.S. Application Data
(63) Continuation of application No. PCT/US01/08998, filed on Mar. 21, 2001.
(60) Provisional application No. 60/191,089, filed on Mar. 22, 2000.

(51) Int. Cl. A01K 67/027; G01N 33/567; G01N 33/53; C12N 9/00

(52) U.S. Cl. 800/21; 435/325; 435/183; 435/7.2

(57) ABSTRACT

The specification described a novel compound, oscillogenin, which is an active agent in furthering oocyte fertilization by sperm or in parthenogenetic activation of an oocyte. The specification discloses methods of isolating oscillogenin to modulate fertility and to enhance parthenogenetic activation of oocytes for nuclear transfer or in ICSI procedures, and methods of using oscillogenin to test amounts of it in sperm and thus sperm fertility

### CROSS REFERENCE TO RELATED APPLICATIONS

This application claims benefit of U.S. provisional application Ser. No. 60/191,089 filed on Mar. 22, 2000, which is hereby incorporated in its entirety by reference.

### FEDERAL FUNDING

This invention was supported in part by Grant No. 99-2371 from the United States Department of Agriculture. The U.S. Government may have rights in the invention.

### FIELD OF THE INVENTION

This invention relates to compositions and methods for parthenogenetic activation of oocytes, modulating sperm fertility and assessing sperm fertility. One composition comprises a sperm protein, oscillogenin.

### BACKGROUND OF THE INVENTION

The fields of animal husbandry and artificial reproductive technology (ART) need improved nuclear transfer techniques to increase the efficiency and success rates of the current methods being used. The benefits obtained from artificial reproductive techniques are numerous. For example, the cloning of embryonic cells, together with the ability to transplant the cloned embryonic cells, allows production of several genetically identical animals. Cloning by nuclear transfer is preferable to other methods (e.g., embryo splitting or embryonic cell aggregation to produce fetal placental chimeras), because it allows for (1) the production of multiple copies of genetically identical animals; (2) the selection of specific traits; and (3) the cryogenic storage of the embryonic cells until completion of testing.

1. Nuclear Transfer

The first successful transfer of a nucleus from an adult mammary gland cell into an enucleated oocyte was reported in 1996 (Campbell et al., Nature 380: 64-6 (1996)). Nuclear transfer (NT) involves preparing a cytoplast as a recipient cell. In most cases, the cytoplast is derived from a mature metaphase II oocyte, from which the chromosomes have been removed. A donor cell nucleus is then placed between the zona and the cytoplast. Fusion and cytoplast activation are initiated by electrical stimulation. Successful reprogramming of the donor cell nucleus by the cytoplast is critical, and is a step which may be influenced by cell cycle (Wolf et al., Biol. Reprod. 60: 199-204 (1999)).

A number of pregnancies have been established using fetal cells as the source of donor nuclei. However, animal cloning is facilitated by the use of cell lines to create transgenic animals, which allow for the genetic manipulation of the cells in vitro before nuclear transfer. Id. The mechanisms regulating early embryonic development may be conserved among mammalian species, such that, for example, a bovine oocyte cytoplasm can support the introduced, differentiated, donor nucleus regardless of chromosome number, species or age of the donor fibroblast (Dominko et al., Biol. Reprod. 60: 1496-1502 (1999)).

Actively dividing fetal fibroblasts can be used as nuclear donors according to the procedure described in Cibelli et al, Science 280: 1256-9 (1998). Additional methods of preparing recipient oocytes for nuclear transfer of donor differentiated nuclei can be performed as described in International PCT Application Nos. 99/05266; 99/01164; 99/01163; 98/3916; 98/30683; 97/41209; 97/07668; 97/07669; and U.S. Pat. No. 5,843,754. Typically the transplanted nuclei are obtained from cultured embryonic stem (ES) cells, embryonic germ (EG) cells or other embryonic cells (See, e.g., International PCT Applications Nos. 95/17500 and 95/10599; Canadian Patent No. 2,092,258; Great Britain Patent No. 2,265,909; and U.S. Pat. Nos. 5,453,366; 5,057,420; 4,994,384; and 4,664,097). Inner cell mass (ICM) cells can also be used as nuclear donors (Sims et al., Proc. Natl Acad. Sci. USA 90: 6143-7 (1990); and Keefer et al., Biol. Reprod. 50: 935-9 (1994).

II. Calcium Induction in Oocytes and Oocyte Activation

Fertilization in manunalian species as well as in other animals is characterized by the presence of calcium ion (Ca²⁺) oscillations, which can last for several hours in mammals (Miyazaki et al., Dev. Biol. 118: 259-67 (1986); Wu et al., Dev. Biol. 203: 369-81 (1998)); Swann et al., J Exp. Zool. 285: 267-75 (1999). Such Ca²⁺ oscillations are necessary to trigger egg activation and initiate embryonic development (Id.), which consists of a sequence of events including cortical granule exocytosis, resumption of meiosis and extrusion of the second polar body, pronuclear formation, DNA synthesis and first mitotic cleavage (Kline et al., Dev. Biol. 149: 80-89 (1992); and Schultz et al., Curr. Topics Dev. Biol. 30: 21-62 (1995)). The mechanisms by which the sperm initiates Ca²⁺ release are unknown (Id.), but three theories are proposed (Swann et al., 1999). First, the sperm acts as a conduit for Ca²⁺ entry into the egg after membrane fusion. Second, the sperm acts on plasma membrane receptors to stimulate a phospholipase C (PLC) within the egg to generate inositol 1,4,5-triphospbate (InSP₃ or IP₃). Lastly, a sperm may induce Ca²⁺ release by a yet unidentified sperm protein. All but the last have been shown not to be primarily responsible for oocyte activation (Wu et al., Dev. Biol. 203: 369-81 (1998)). IP₃ mediates Ca²⁺ release by interacting with IP₃ receptors (IP₃R), which are localized in the endoplasmic reticulum and form tetrameric complexes (Patel et al., Cell Calcium 25: 247-64 (1999)). Injection of GTPγ[S], a non-hydrolyzable activator of G-proteins and consequently of PLC, induced repetitive Ca²⁺ responses in eggs of several species, demonstrating that this pathway is functional in mammalian eggs (Miyazaki, J. Cell. Biol. 106: 345-53 (1988); and Fissore et al., Biol. Reprod. 53: 766-74 (1995)). Furthermore, injection of IP₃ has also been shown to induce Ca²⁺ release in mammalian eggs (Miyazaki et al., 1988; Schultz et al., 1995).

There are three defined isoforms of the IP₃R expressed in mammalian eggs (Fissore et al., Biol. Reprod. 60: 49-57 (1999); and He et al., Biol. Reprod. 61: 935-43 (1999)), although IP₃R subtype I (IP₃R-1) is expressed abundantly and in overwhelmingly larger amounts than the other isoforms (Parrington et al., Dev. Biol. 203: 451-61 (1998); and He et al., Biol. Reprod. 57: 1245-55 (1997)). Also, the IP₃R-1 protein is expressed in mammalian eggs in a stage-specific manner, suggesting an important role in fertilization. For instance, less than 20 mouse and bovine eggs are required to detect the IP₃R-1 protein by Western blotting (He et al., 1997; Fissore et al., 999), and the amounts of IP₃R-1 protein increase significantly during oocyte maturation (Mehlmann et al., Dev. Biol. 180: 489-98 (1996); and He et al., 1997). This increase in receptor density results in an increased IP₃R responsiveness during oocyte maturation (Fujiwara et al., Dev. Biol. 156: 69-79 (1993); and Mehlmann et al., Biol. Reprod. 51: 1088-98 (1994)). Furthermore, injection of the blocking IP₃R-1 monoclonal antibody 18A10 prior to insemination inhibited, in a dose-dependent manner, fertilization-associated Ca²⁺ release and activation in mouse eggs (Miyazaki et al., Science 257: 251-5 (1992); and Xu et al., Development 120: 1851-9 (1994)).

Ca²⁺ release through the IP₃R system may be controlled, in addition to several other mechanisms by regulating the levels of the IP₃R-1 protein. Studies in somatic cell lines have shown that IP₃R down-regulation follows persistent stimulation of IP₃ production induced by activation of cell surface receptors coupled to PLC (Wojcikiewicz et al., J. Biol. Chem 269: 7963-9 (1994); Wojcikiewicz et al., J. Biol. Chem. 270: 11678-83 (1995); and Sipma et al., Cell Calcium 23: 11-21 (1998)). This degradation of IP₃R, which led to decreased cellular responsiveness to IP₃, was shown to be specific since it was not accompanied by general protein degradation (Wojcikiewicz et al., J. Biol. Chem. 271: 16652-5 (1996); and Bokkala et al., J. Biol. Chem. 272: 12454-61 (1997)), was associated with IP₃-binding to the IP₃R (Zhu et al., J. Biol. Chem. 274: 3476-84 (1999)), and was mediated by the proteasome, a multi-protein cellular complex involved with degradation of ubiquinated proteins (Bokkala et al., 1997; Oberdorf et al., Biochem. J. 339: 453-61 (1999)). During fertilization, mammalian eggs also exhibit decreased IP₃R responsiveness as they progress to the pronuclear stage (Fissore et al., Dev. Biol. 166: 634-42 (1994); Jones et al., Development 121: 3259-66 (1995); and Machaty et al., Biol. Reprod. 56: 921-30 (1997)) and this appears to be accompanied by IP₃R-1 down-regulation (Parrington et al., 1998; and He et al., Biol. Reprod. 61: 935-43 (1999)). However, the mechanism(s) that controls the demise of IP₃R-1 in mammalian eggs is not known. Moreover, parthenogenetic activation of mammalian eggs, the use of which has become widespread with the advent of cloning techniques, can be induced by several agonists that stimulate single or multiple Ca²⁺ rises, but their effects on IP₃R-1 numbers have not been determined. Thus, we investigated the signaling mechanism that controls IP₃R-1 down-regulation in mouse eggs including the possible involvement of the proteasome pathway.

Sperm cytosolic factors are necessary for oocyte activation (Stice et al., Mol. Reprod. Dev. 25: 272-80 (1990) and Swann et al., Devel. 110: 1295-302 (1990)). Activation of mammalian oocytes involves exit from meiosis and entry into the mitotic cell cycle by the secondary oocyte, and the formation and migration of pronuclei within the cell. Thus, oocyte activation requires cell cycle transitions. Although fertilization (U.S. Pat. No. 5,496,720) and a sperm's cytoplasmic fraction (Swann et al., 1990) can induce Ca²⁺ oscillations, activation can also be induced by parthenogenic treatments that induce single or multiple Ca²⁺ oscillations. Parthenogenetic activation may be used to prepare the oocytes for nuclear transfer.

Parthenogenesis is the production of embryonic cells, with or without eventual development into an adult, from a female gamete in the absence of any contribution from a male gamete (U.S. Pat. No. 5,496,720). Parthenogenetic activation of mammalian oocytes can be performed by (1) use of electric shock, electroporation or electrical stimulation; (2) combined treatment with ionomycin and 6-dimethylaminopurine (DMAP); (3) combined treatment with the calcium ionophore A23187 and 6-DMAP (Susko-Parrish et al., Dev. Biol. 166: 729-39 (1994); Mitalipov et al., Biol. Reprod. 60: 821-7 (1999); Liu et al., Biol Reprod. 61: 1-7 (1999); and U.S. Pat. No. 5,496,720). The latter two methods use calcium ionophores in combination with protein kinase inhibitors, which are important for inducing protein kinase inhibitor release (Mayes et al., Biol. Reprod. 53: 270-5 (1995)).

Other divalent cations utilized for oocyte activation include magnesium, strontium, barium or calcium, e.g., in the form of an ionophore. Divalent cation levels can also be increased by means of electric shock, oocyte treatment with ethanol, and treatment with caged chelators. Phosphorylation in oocytes may be reduced by addition of kinase inhibitors (e.g., serine-threonine kinase inhibitors, such as 6-dimethylaminopurine, staurosporine and sphingosine) (U.S. Pat. No. 5,945,577). Alternatively, oocyte protein phosphorylation may be inhibited by introducing a phosphatases into the oocyte (e.g., phosphatase 2A and phosphatase 2B) (Id.).

Alternatively, activation may be achieved by application of known activation agents. For example, penetration of oocytes by sperm during fertilization has been shown to yield greater numbers of viable pregnancies and multiple genetically identical calves after nuclear transfer. Also, treatment with electrical and chemical shock may be used to activate NT embryos after fusion. Suitable oocyte activation methods are the subject of U.S. Pat. No. 5,496,720, to Susko-Parrish et al., herein incorporated by reference in its entirety.

Oscillin. Several groups postulated that sperm activates oocytes via a protein which induces Ca²⁺ oscillation. The putative proteins were termed oscillogen (Panington et al., Nature 379: 364-8 (1996)). Oscillin was the first identified oscillogens, and was believed to induce intracellular calcium release in oocytes (Id.). Oscillin in fact is glucosamine 6-phosphate dearninase (Wolosker et al., FASEB J. 12: 91-9 (1998)). However, despite experiments that purportedly demonstrated that oscillogen induced oocyte activation to the same extent as oocyte injection with a spermatid nuclei (Sasagawa et al., J. Urol. 158: 2006-8 (1997); Wolny et al., Mol. Reprod. & Dev. 52: 277-87 (1999)), oscillin was later demonstrated not to be the sperm protein responsible for Ca²⁺ release in oocytes (Wolosker et al., (1998); and Wu et al., Dev. Biol. 203: 369-81 (1998)). As a consequence, the sperm factor responsible for oocyte activation remains unknown (Wolny et al., 1999).

III. Preparing Somatic Cells for Nuclear Transplantation or Nuclear Transfer

For purposes of animal husbandry, nuclear transfer can be used with embryonic stem cells (ES), inner cell mass cells (ICMs) and somatic cells.

Embryonic Stem Cells. Another system for producing transgenic animals has been developed that uses ES cells. In mice, ES cells have enabled researchers to select for transgenic cells and perform gene targeting. This method allows more genetic engineering than is possible with other transgenic techniques. For example, ES cells are relative-ly easy to grow as colonies in vitro, can be transfected by standard procedures, and the transgenic cells clonally selected by antibiotic resistance (Doetschman, “Gene transfer in embryonic stem cells.” IN TRANSGENIC ANIMAL TECHNOLOGY: A LABORATORY HANDBOOK 115-146 (C. Pinkert, ed., Academic Press, Inc., New York 1994)). Furthermore, the efficiency of this process is such that sufficient trans-genic colonies (hundreds to thousands) can be produced to allow a second selection for homologous recombinants (Id.). ES cells can then be combined with a normal host embryo and, because they retain their potency, can develop into all the tissues in the resulting chimeric animal, including the germ cells. Thus, the transgenic modification is transmissible to subsequent generations.

Methods for deriving embryonic stem (ES) cell lines in vitro from early preimplantation mouse embryos are well known (Evans et al., Nature 29: 154-6 (1981); and Martin, Proc. Natl. Acad. Sci. USA 78: 7634-8 (1981)). ES cells can be passaged in an undifferentiated state, provided that a feeder layer of fibroblast cells (Evans et al., 1981) or a differentiation inhibiting source (Smith et al., Dev. Biol. 121: 1-9 (1987)), is present.

In view of their ability to transfer their genome to the next generation, ES cells have potential utility for germ line manipulation of livestock animals. Some research groups have reported the isolation of pluripotent embryonic cell lines. For example, Notarianni et al., J. Reprod. Fert. Suppl. 43: 55-260 (1991) reported the establishment of stable, pluripotent cell lines from pig and sheep blastocysts, which exhibit some morphological and growth characteristics similar to that of cells in primary cultures of inner cell masses (ICMs) isolated immunosurgically from sheep blastocysts. Also, Notarianni et al., J. Reprod. Fert. Suppl. 41: 51-56 (1990) disclosed maintenance and differentiation in culture of putative pluripotent embryonic cell lines from pig blastocysts. Gerfen et al., Anim. Biotech. 6: 1-14 (1995) disclosed the isolation of embryonic cell lines from porcine blastocysts, which do not require mouse embryonic fibroblast feeder layers and reportedly differentiate into several different cell types during culture.

Further, Saito et al., Roux's Arch. Dev. Biol. 201: 134-41 (1992) reported cultured, bovine embryonic stem cell-like cell lines, which survived three passages, but were lost after the fourth passage. Handyside et al., Roux's Arch. Dev. Biol. 196: 185-90 (1987) disclosed culturing immunosurgically isolated sheep embryo ICMs under conditions that allow for the isolation of mouse ES cell lines derived from mouse ICMs.

Campbell et al., Nature 380: 64-6 (1996) reported the production of live lambs following nuclear transfer of cultured embryonic disc (ED) cells from day nine ovine embryos cultured under conditions which promote the isolation of ES cell lines in the mouse.

Purportedly, animal stem cells have been isolated, selected and propagated for use in obtaining transgenic animals (see Evans et al., WO 90/03432; Smith et al., WO 94/24274; and Wheeler et al., WO 94/26884). Evans et al. also reported the derivation of purportedly pluripotent ES cells from porcine and bovine species, which purportedly are useful for the production of transgenic animals.

ES cells from a transgenic embryo can be used in nuclear transplantation. The use of ungulate ICM cells for nuclear trans-plantation also has been reported. In the case of live-stock animals (e.g., ungulates) nuclei from similar preimplantation livestock embryos support the development of enucleated oocytes to term (Keefer et al., Biol. Reprod. 50: 935-39 (1994); Smith et al., Biol. Reprod. 40: 1027-1035 (1989)). In contrast, nuclei from mouse embryos do not support development of enucleated oocytes beyond the eight-cell stage after transfer (Cheong et al., Biol. Reprod. 48: 958-63 (1993)). Therefore, ES cells from livestock animals are highly desirable, because they may provide a potential source of totipotent donor nuclei, genetically manipulated or other-wise, for nuclear transfer procedures.

Use of ICM Cells. Collas et al., Mol. Reprod. Dev. 38: 264-7 (1994) disclosed nuclear transplantation of bovine ICMs by microinjection of the lysed donor cells into enucleated mature oocytes. Culturing of embryos in vitro for seven days produced fifteen blastocysts which, upon transfer into bovine recipients, resulted in four pregnancies and two births. Also, Keefer et al. (1994) disclosed the use of bovine ICM cells as donor nuclei in nuclear transfer procedures, to produce blastocysts which also resulted in several live offspring. Further, Sims et al., Proc. Natl. Acad. Sci. USA 90: 6143-7 (1993) disclosed the production of calves by transfer of nuclei from short-term in vitro cultured bovine ICM cells into enucleated mature oocytes.

IV. Intracvtoplasmic Sperm Injection (ICSI)

Sperm can be obtained by one of several methods including microsurgical epidiymal sperm aspiration (MESA) and testicular sperm extraction (TESE). In instances of mature epidiymal spenmatozoa and testicular spermatozoa, when injected into mature mouse oocytes, normal embryo development and resulting mice occur (Sasagawa et al., J. Urol. 158: 2006-8 (1997)). However, round spermatids are unable to activate oocytes (Id.). Therefore, it for purposes of animal husbandry as well as for artificial reproductive techniques, the simultaneous injection of oscillogenin into oocytes can be used to initiate normal embryo development when using immature sperm or round spernatids.

ICSI is a technique developed for use in artificial reproduction and in vitro fertilization in the ART field to assist men with defective sperm. Some have suggested that this procedure has revolutionized the treatment of male infertility, as normal fertilization can not be achieved with severely affected spermatozoa (Tarlatzis et al., Hum. Reprod. 13S: 165-77 (1998)). For example, cystic fibrosis has been suggested to cause congenital aphasia of the vas deferens, which reduces sperm quality (Jakubiczka et al., Hum. Reprod. 14: 1833-4 (1999)). Other causes of male infertility include Y-chromosomal microdeletions leading to spermatogenic impairment and karyotype abnormalities (Kim et al., Prenat. Diagn. 18: 1349-65 (1998)). Sperm effectiveness can also be decreased as a result of exposure to protamine (Ahmadi et al., J. Assist. Reprod. Genet. 16: 128-32 (1999)). ICSI is also relevant to animal husbandry (see, e.g., Li et al., Zygote 7: 233-7 (1999)).

Gomez et al., Reprod. Fertil. Dev. 10: 197-205 (1998), suggested that the presence of calcium in the media enhanced fertilization rates after ICSI. This was not unexpected, as Sousa et al., Mol. Hum. Reprod. 2: 853-7 (1996), suggested that a soluble sperm factor (SSF) was likely responsible for the Ca²⁺ oscillations driving oocyte activation after ICSI. The Ca²⁺ wave may be large enough to generate all the responses associated with fertilization (Iranga et al., Int'l. J Dev. Biol. 40: 515-9 (1996)). Additionally, the absence of the typical oscillatory Ca²⁺ response in spermatocyte-injected oocytes is presumed to be due to the actual deficiency of SSF in the spennatocytes, rather than to defective responsiveness of the injected oocytes or to the failure of SSF release into the oocyte cytoplasm (Sousa el al. (1996)). Additionally, the calcium response may be important for normal embryonic development after spermatid conception (Id.). Tesarik et al., Biol. Reprod. 51: 385-91 (1994) reported that although Ca²⁺ oscillations are observed in ICSI fertilized oocytes, it occurs only after a considerable delay. 5 ICSI is also used in techniques to karyotype human spermatozoa with poor fertilization capacity of sperm. For, example, Goud et al., Hum. Reprod. 13: 1336-45 (1998) discusses techniques and conditions for assessing parthenogenetic activation of Syrian golden hamster oocytes microinjected with human spermatozoa.

Sperm fertility also can be assessed using antibodies directed to heparin binding proteins and assessing the heparin binding protein content of sperm membranes (U.S. Pat. No. 5,962,241). Using the sperm factor, oscillogenin, described herein, may help to overcome certain growth abnormalities which may result from the manipulation of preimplantation embryos in vitro (e.g., large calf syndrome) caused perhaps by genetic imprinting (Moore et al., Rev. Reprod. 1: 73-7 (1996)) or perhaps reducing the high lethality of reconstructed embryos. Genetic imprinting is related to protein kinase activity, which in turn may be controlled, in part by the calcium ion oscillations observed upon normal sperm-induced fertilization of a mature oocyte. Such developmental abnormalities can be detrimental and even lethal to the afflicted animal. Additional methods of determining sperm fertility are discussed in U.S. Pat. Nos. 5,770,363; 5,763,206; 5,434,057; and 5,358,847.

Therefore, notwithstanding what has previously been reported in the literature, there exists a need for im-proved methods of inducing oocytes Ca²⁺ oscillations for activating oocytes, especially for use with nuclear transfer and ICSI and other related artificial reproductive technologies. Additionally, methods of making and using oscillogenin and agents regulating oscillogenin will greatly aid the production of cloned livestock, the use of ART for the birth of healthy humans, and for contraception.

### OBJECTS AND SUMMARY OF THE INVENTION

It is an object of the invention to provide a novel method of isolating oscillogenin from sperm comprising: (A) preparing a sperm cytoplasmic fraction; (B) isolating oscillogenin by sequentially processing the sperm cytoplasmic fraction through a HiTrap blue affinity FPLC chromatographic column, a hydroxyapatite FPLC column, and a surperose 12 FPLC chromatographic column; and (C) obtaining a fraction with [Ca²⁺]₁ releasing activity.

It is another object of the invention to provide a method of enhancing oocyte activation of an oocyte comprising the step of injecting oscillogenin into an oocyte prior to, simultaneously with, or immediately after injecting or fusing the oocyte with a sperm or other cell nuclei, wherein said oocyte has been treated, before or after oscillogenin injection, to remove or inactivate its endogenous nucleus. The oocyte can be a mamnmalian oocyte (e.g., primate, bovine, caprine, ovine, porcine, feline, murine, or canine), and may be preferably a human oocyte. The method may further comprise the step of injecting the oocyte with at least one agent which additionally enhances divalent cation release or a combination of such agents.

It is a more specific object of the invention to allow the activated oocyte to develop into an embryo, and in some circumstances, this embryo may be implanted into a female surrogate and allowed to gestate into a non-human animal.

It is another object of the invention to provide a method of enhancing intracytoplasmic sperm injection (ICSI) comprising the step of injecting an oocyte with oscillogenin either before or after a sperm or sperm nucleus is inserted into the oocyte. Another objection of the invention is to provide a method of enhancing parthenogenetic activation of an oocyte comprising the step of injecting an oocyte with oscillogenin.

It is another object of the invention to provide a method of predicting sperm [Ca²⁺]ᵢ releasing activity comprising measuring oscillogenin concentration in a sperm sample. It is a more specific object to also provide a kit for predicting sperm [Ca²⁺]ᵢ releasing activity comprising a labeled agent which recognizes and binds to oscillogenin or a nucleic acid encoding oscillogenin.

It is a further object of the invention to provide a nucleic acid encoding an oscillogenin, as well as its corresponding amino acid sequence. The oscillogen sequence can be human, primate, bovine, porcine, ovine, equine, feline, canine, murine and caprine.

Another object of the invention provides an antibody or immunogenic fragment thereof which recognizes and binds to oscillogenin. Preferably the antibody is a monoclonal antibody and the immunogenic fragment is consisting of: Fab, scFv, F(ab′)₂ and Fab′.

### BRIEF DESCRIPTION OF THE FIGURES

FIG. 1. Panel A shows the step-wise use of chromatographic columns utilized to obtain elutions enriched in sperm factor (SF) proteins. Sperm fractions are first processed through a HiTrap blue dye column, then followed by a hydroxyapatite column and lastly by a Superose 12 column. Panel B shows the distribution of proteins in the various fractions (F1-F5) separated using polyacrylamide gel electrophoresis after processing through the various chromatographic columns. Panel C shows the [Ca²⁺]ᵢ releasing activity of each of the fractions from the multi-column. The greatest activity monitored is in the F4-1 fraction.

FIG. 2. Western blot analysis and quantification of IP₃R-1 immunoreactivity in mouse eggs aged in vitro (A and B) or following in vivo fertilization C and D). Twenty eggs (e) were used in each lane. “UF” stands for unfertilized and “F” for fertilized eggs. MII eggs were always at 16 hr post human chorionic gonadotrophin (“hCG”) and the indicated times refer to hr post hCG (phCG). Data are presented as means±SEM. Treatments under bars that share a common superscript are not significantly different (p>0.05). Values are the mean of 4 Western-blotting experiments, performed on different batches of eggs.

FIG. 3. Western blot analysis and quantification of IP₃R-1 immunoreactivity in mouse eggs activated by exposure to 7% ethanol (Et; A and B) or ionomycin/DMAP (Io/D; C and D). Activation was started at 16 hr phCG. Et-24 hr-1-cell were those eggs that at 24 hr phCG exhibited pronuclear formation after ethanol exposure; and Et-24 hr-2 cell were those that divided into two-cells within 4 hr of exposure. All eggs at 24 hr phCG (8 hr post activation) for all treatments exhibited pronuclear formation. Twenty eggs (e) were used per lane. Treatments under bars with different superscripts are significantly different (p<0.05). Values are the mean of three Westem-blotting experiments, performed on different batches of eggs.

FIG. 4. Western blot analysis and quantification of IP₃R-1 immunoreactivity in mouse eggs activated by injection of SF (A and B) or adenophostin A (Ad; C and D). Injections were carried out at 16 hr phCG and samples taken within 1 hr, 2 hr, 4 hr, and 8 hr post-injection. Treatments under bars with different superscripts are significantly different (p<0.05). Values are the mean of three Westem-blotting experiments, performed on different batches of eggs.

FIG. 5. Western blot analysis and quantification of IP₃R-1 immunoreactivity in mouse eggs activated by SrCl₂ (A and B) or thimerosal (“Th”; C and D). Activation was carried out at 16 hr phCG and 15 eggs were used per lane. “C” stands for control and these eggs were cultured for the same amount of time but were not exposed to SrCl₂. Treatments under bars that do not share common superscripts are significantly different (p<0.05). Values are the mean of five Western-blotting experiments, performed on different batches of eggs.

FIG. 6. [Ca²⁺]ᵢ oscillations profiles in mouse eggs induced by several of the agonists used in this study. Injection of SF (˜10 ng/μl intracellular concentration) induced highly repetitive rises (A) similar to those induced by injection of adenophostin A (B; ˜100 nM intracellular concentration). SrCl₂ induced prolonged rises and of slower frequency (C). Thimerosal, which was incubated with eggs for 30 min, induced repetitive rises (D). The presented Ca²⁺ recordings were carried out in three separate experiments, and the termination of the oscillations was due to the termination of the recordings rather than to cessation of the oscillations.

FIG. 7. Western blot analysis and quantification of IP₃R-1 immunoreactivity in mouse eggs activated by SF in the presence or absence of lactacystin (“Lac”). Injection of SF was carried out at 16 hr phCG. Eggs were preincubated with the inhibitor (100 μM) for 30 min. prior to SF injection and were cultured in Lac for 2 hr after the injections. Fifteen eggs (e) were used per lane. Treatments under bars that do not share common superscripts are significantly different (p<0.05). Values are the mean of 3 Western blotting experiments, performed on different batches of eggs.

### DETAILED DESCRIPTION OF THE INVENTION

This invention is directed towards methods of isolating oscillogenin from sperm as well as recombinant production of oscillogenin. The protein can then be used to enhance parthenogenetic activation of oocytes and enhance sperm fertility. Detection of the protein or nucleic acids which encode the protein can be used to assess sperm fertility. The invention also considers modulation of oscillogenin activity to thereby regulate fertility.

I. Definitions

By “oscillogenin” is meant the protein responsible for Ca²⁺ oscillations when injected into an unactivated oocyte. In the instance of oscillogenin purified from sperm cell extracts, the “purified oscillogenin” is obtained by sequentially processing the oscillogenin through at least three chromatographic columns and obtaining the [Ca²⁺]ᵢ releasing fractions. Said fractions will comprise oscillogenin and about five other proteins as assessed by silver staining. More preferred purified oscillogenin compositions will comprise oscillogenin and about three other proteins as determined by silver staining. The preferred sequential chromatographic columns used is as described in Example 1. By “sequentially processing” is meant the columns used preferably in the order described in Example 1.

By “nucleic acid” or “nucleic acid molecule” is meant to include a DNA, RNA, mRNA, cDNA, or recombinant DNA or RNA.

By “animal” is meant any member of the animal kingdom including vertebrates (e.g., frogs, salamanders, chickens, or horses) and invertebrates (e.g., worms, etc.). Preferred animals are mammals. Preferred mammalian animals include livestock animals (e.g., ungulates, such as bovines, buffalo, equines, ovines, porcines and caprines), as well as rodents (e.g., mice, hamsters, rats and guinea pigs), canines, felines and primates. By “non-human” is meant to include all animals, especially mammals and including primates other than human primates.

By “female surrogate” is meant a female animal into which an embryo of the invention is inserted for gestation. Typically, the female animal is of the same animal species as the embryo, but the female surrogate may also be of a different animal species. The embryo, as used herein, can include a complex of two or more cells.

By “cytoplast” is meant the fragment of the cell remaining once the nucleus is removed.

By “parthenogenetic activation” is meant development of an ovum or oocyte without fusion of its nucleus with a male nucleus or male cell to form a zygote.

By “oocyte” is meant an animal egg, nucleated or enucleated which has not undergone a Ca²⁺ oscillations.

By “activated oocyte” is meant an oocyte which acts as though it has been parthenogenically activated or as though it has been fertilized.

By “enucleated oocyte” is meant an animal egg which has had its endogenous nucleus removed or inactivated.

By “sperm,” “semen,” “sperm sample,” and “semen sample” are meant the ejaculate from a male animal which contains spernatozoa. A mature sperm cell is a “spermatozoon,” whereas the precursor is a “spermatid.” Spermatids are the haploid products of the second meiotic division in spermatogenesis, which differentiate into spermatozoa.

By “sperm fertility” is meant the ability of a sperm to fertilize an egg and create an embryo. By “sperm [Ca²⁺]ᵢ-releasing activity” is meant the ability of a sperm to activate an oocyte (of any animal), which can be measured by induction of Ca²⁺ oscillations in the oocyte.

By “sperm cytoplasmic fraction” is meant the portion of the cell which lacks the nucleus and most of the genetic material. Preferably, the cytoplasm fraction comprises the substances contained within the plasma membrane but excluding the nucleus and its genetic material.

By “inducing”, “increasing,” “enhancing” or “up-regulating” is meant the ability to raise the level of oscillogenin activity. By “enhancing activation” is meant a method or agent which increases oocyte activation.

By “modulating” or “regulating” is meant the ability of an agent to alter.(e.g., up-regulate or down-regulate) from the wild type level observed in the individual organism the activity of oscillogenin. Oscillogenin activity can be at the level of transcription, translation, nucleic acid or protein stability or protein activity.

By “antibody fragment” and “immunogenic fragment” is meant an immunogenic protein peptide capable of recognizing and binding to oscillogenin or a fragment thereof. This includes an anti-oscillogenin antibody or polypeptide fragment thereof.

By “intracytoplasmic sperm injection” or “ICSI” is meant injection of a sperm or at least the genetic contents of a sperm into an oocyte.

The terms “nuclear transfer” or “nuclear transplantation” refer to a method of cloning, wherein the donor cell nucleus is transplanted into a cell before or after removal of its endogenous nucleus. The cytoplast could be from an enucleated oocyte, an enucleated ES cell, an enucleated EG cell, an enucleated embryonic cell or an enucleated somatic cell. Nuclear transfer techniques or nuclear transplantation techniques are known in the literature (Campbell et al., Theriogenology 43: 181 (1995); Collas et al., Mol. Reprod. Dev. 38: 264-267 (1994); Keefer et al., Biol. Reprod. 50: 935-939 (1994); Sims et al., Proc. Natl. Acad. Sci. USA 90: 6143-6147 (1993); Evans et al., WO 90/03432; Smith et al., WO 94/24274; and Wheeler et al., WO 94/26884. Also U.S. Pat. Nos. 4,994,384 and 5,057,420 describe procedures for bovine nuclear transplantation. In the subject application, “nuclear transfer” or “nuclear transplantation” or “NT” are used interchangeably.

The terms “nuclear transfer unit” and “NT unit” refer to the product of fusion between or injection of a somatic cell or cell nucleus and an enucleated cytoplast (e.g., an enucleated oocyte), which is some-times referred to herein as a fused NT unit.

By “somatic cell” is meant any cell of a multicellular organism, preferably an animal, that does not become a gamete.

By “isolated” or “purified” oscillogenin is meant a Ca²⁺-activity protein substantially purified from either the sperm it is isolated from or the cell used to recombinantly prepare the oscillogenin protein or peptide from other non-oscillogenin proteins, peptides or nucleic acids.

By “protein kinase inhibitor” is an agent which inhibits an enzyme that catalyzes the transfer of phosphate from ATP to hydroxyl side chains on proteins causing changes of function of the protein. The preferred protein kinase inhibitors of this invention are 6-dimethylaminopurine (DMAP), staurosporine, butyrolactone, roscovitine, p34(cdc2) inhibitors, 2-aminopurine and sphingosine.

By “phosphatase” is meant an enzyme that hydrolyzes phosphomonoesters. The preferred phosphatases described herein are phosphatase 2A and 2B.

By “calcium ionophore” are agents which allow calcium ions (Ca⁺²) to cross lipid bilayer. Preferred calcium ionophores include ionomycin and A23187.

By “differentiate” or “differentiation” is meant to refer to the process in development of an organism by which cells become specialized for particular functions. Differentiation requires that there is selective expression of portions of the genome.

By “inner cell mass” or “ICM” is meant a group of cells found in the mammalian blastocyst that give rise to the embryo and are potentially capable of forming all tissues, embryonic and extra-embryonic, except the trophoblast.

By “feeder layer” is meant a layer of cells to condition the medium in order to culture other cells, particularly to culture those cells at low or clonal density.

By “medium” or “media” is meant the nutrient solution in which cells and tissues are grown.

The term “pharmaceutically acceptable carrier”, as used herein means a pharmaceutically-acceptable material, composition or vehicle, such as a liquid or solid filler, diluent, excipient, solvent or encapsulating material, involved in carrying or transporting a chemical agent. The diluent or carrier ingredients should not be such as to diminish the therapeutic effects of the active compound(s).

The term “composition” as used herein means a product which results from the mixing or combining of more than one element or ingredient.

II. Method of Isolating Oscillopenin from Sperm

Sperm fractions with oscillogenin can be obtained from animal sperm by first preparing cytosolic sperm extracts as described by Wu et al., Mol. Reprod. Devol. 46: 176-89 (1997) and Wu et al., Mol. Reprod. Dev. 49: 37-47 (1998). Briefly, semen samples are washed twice with TL-Hepes medium, and the sperm pellet is resuspended in a solution containing 75 mM KCl, 20 mM Hepes, 1 mM ethylenediaminetetraacetic acid (EDTA), 10 mM glycerophosphate, 1 mM DTT, 200 μM PMSF, 10 μg/ml pepstatin, 10 μg/ml leupeptin, at pH 7.0. The sperm suspension is sonicated for about 25 to 35 minutes at 4° C. (XL2020, Heat Systems, Inc., Farmingdale, N.Y.). The lysate is then spun twice at 10,000×g and, the supernatants collected. The resulting supernatant is then centrifuged at 100,000×g for one hour at 4° C. This clear supernatant represents the cytosolic fraction of the sperm. Active sperm fractions-can also be obtained from, for example, pig sperm by freezing and thawing the sperm twice in the absence of a cryoprotectant.

The cytosolic fraction thus obtained is then subjected ammonium sulfate precipitation (50%) and precipitated. The precipitate can then be pelleted by centrifugation and stored at −20 to −80° C. for prolonged periods of time. The pellet can be reconstituted and subjected to the following chromatographic procedures for isolation and/or purification of oscillogenin. The reconstituted pellet is first subjected to a hydroxyapatite FPLC chromatographic column, followed by a chromatofocusing column, and then followed by a Superose 12 FPLC chromatographic column. The fraction eluting at a molecular weight of approximately 30 to about 68 kDa contains the [Ca²⁺]ᵢ-inducing agent, oscillogenin. The conditions for of the chromatographic columns can be used as described for the individual chromatographic columns used in Wu et al., 1998. Wu et al., (1998) do not describe the specific sequential use of the chromatography columns as described herein, nor do Wu et al. describe which specific fractions are to be used that contain the activation factor(s).

III. Characterization of Oscillogenin

Once the oscillogenin is isolated from sperm, it can be peptide sequenced. Using the peptide sequences thus identified, degenerate probes can be created which can be used to screen libraries to identify the gene, which encodes oscillogenin.

The present invention further provides nucleic acid molecules that encode oscillogenin and related proteins, preferably in isolated form. As used herein, “nucleic acid” is defined as RNA, rRNA, mRNA, DNA, rDNA or cDNA sequences which encode oscillogenin or a polypeptide fragment thereof, or is complementary to nucleic acid sequence encoding oscillogenin or a polypeptide fragment thereof, or hybridizes to such a nucleic acid and remains stably bound to it under appropriate stringency conditions, or encodes a polypeptide sharing at least 75% sequence identity, or preferably at least 80%, or more preferably at least 85%, or most preferably at least about 90-95% identify with the peptide sequences. Specifically contemplated are genomic DNA, cDNA, MRNA and antisense molecules, as well as nucleic acids based on alternative backbone or including alternative bases whether derived from natural sources or synthesized. Such hybridizing or complementary nucleic acids, however, are defined further as being novel and nonobvious over any prior art nucleic acid including that which encodes, hybridizes under appropriate stringency conditions, or is complementary to a nucleic acid encoding an oscillogenin according to the present invention. “Stringent conditions” are those that (1) employ low ionic strength and high temperature for washing, for example, 0.015 M NaCl, 0.0015 M sodium titrate, 0.1% SDS at 50° C.; or (2) employ during hybridization a denaturing agent such as formamide, for example, 50% (vol/vol) formarnide with 0.1% bovine serum albumin, ⁰.¹I/Q Ficoll, 0.1% polyvinylpyrrolidone, 50 mM sodium phosphate buffer at pH 6.5 with 750 mM NaCl, 75 mM sodium citrate at 42° C. Another example is use of 50% formamnide, 5×1452 SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2× SSC and 0.1% SDS. A skilled artisan can readily determine and vary the stringency conditions appropriately to obtain a clear and detectable hybridization signal or use materials and methods as described in MANIATIS ET AL., MOLECULAR CLONING: A LABORATORY MANUAL (1989).

As used herein, a nucleic acid molecule is said to be “isolated” or “purified” when the nucleic acid molecule is substantially separated from contaminant nucleic acids encoding other polypeptides.

The present invention further provides fragments of the encoding nucleic acid molecule. As used herein, a “fragment of an encoding nucleic acid molecule” refers to a small portion of the entire protein encoding nucleic acid sequence. The size of the fragment will be determined by the intended use. For example, if the fragment is chosen to encode an active portion of the protein, the fragment will need to be large enough to encode one or more biologically active region(s) of the protein. If the fragment is to be used as a nucleic acid probe or PCR primer, then the fragment length is chosen so as to obtain a relatively small number of false positives during probing/priming.

Fragments of the encoding nucleic acid molecules of the present invention (i.e., synthetic oligonucleotides) that are used as probes or specific primers for the polymerase chain reaction (PCR), or to synthesize gene sequences encoding proteins of the invention can easily be synthesized by chemical techniques, for example, the phosphotriester method of Matteucci et al., (J. Am. Chem. Soc. 103: 3185-91 (1981)) or using automated synthesis methods. In addition, larger DNA segments can readily be prepared by well known methods, such as synthesis of a group of oligonucleotides that define various modular segments of the gene, followed by ligation of oligonucleotides to build the complete modified gene.

The enicoding nucleic acid molecules of the present invention may further be modified so as to contain a detectable label for diagnostic and probe purposes. A variety of such labels are known in the art and can readily be employed with the encoding molecules herein described. Suitable labels include, but are not limited to, biotin, radiolabeled nucleotides and the like. A skilled artisan can employ any of the art known labels to obtain a labeled nucleic acid molecule.

Modifications to the primary structure itself by deletion, addition, or alteration of the amino acids incorporated into the protein sequence during translation can be made without destroying the activity of the protein. Such substitutions or other alterations result in proteins having an amino acid sequence encoded by a nucleic acid falling within the contemplated scope of the present invention.

Essentially, a skilled artisan can readily use the amino acid sequence of oscillogenin to generate antibody probes to screen expression libraries prepared from appropriate cells. Typically, polyclonal antiserum from mammals such as rabbits immunized with the purified protein (as described below) or monoclonal antibodies can be used to probe a mammalian cDNA or genomic expression library, such as λgtll, to obtain the appropriate coding sequence for other members of the protein family. The cloned cDNA sequence can be expressed as a fusion protein, expressed directly using its own control sequences, or expressed by constructions using control sequences appropriate to the particular host used for expression of the enzyme.

Alternatively, a portion of the coding sequence herein described can be synthesized and used as a probe to retrieve DNA encoding a member of the oscillogenin family of proteins from any organism. Oligomers containing approximately about 18-20 nucleotides (encoding about a 6-7 amino acid stretch) are prepared and used to screen genomic DNA or cDNA libraries to obtain hybridization under stringent conditions or conditions of sufficient stringency to eliminate an undue level of false positives. Oligomers can also be prepared which encode about 8, 9, 10, 15 or more consecutive amino acids of oscillogenin.

Additionally, pairs of oligonucleotide primers can be prepared for use in a polymerase chain reaction (PCR) to selectively clone an encoding nucleic acid molecule. A PCR denature/anneal/extend cycle for using such PCR primers is well known in the art and can readily be adapted for use in isolating other oscillogenin encoding nucleic acid molecules, or as described in NEWTON ET AL., PCR (1997).

Recombinant Oscillogenin. The present invention firther provides recombinant DNA molecules (rDNAs) that contain a coding sequence. As used herein, a rDNA molecule is a DNA molecule that has been subjected to molecular manipulation in situ. Methods for generating rDNA molecules are well known in the art, for example, see SAMBROOK ET AL., CLONING: A LABORATORY MANUAL (1989). In the preferred rDNA molecules, a coding DNA sequenceris operably linked to expression control sequences and/or vector sequences.

The choice of vector and/or expression control sequences to which an oscillogenin encoding sequence is operably linked depends directly, as is well known in the art, on the functional properties desired, e.g., protein expression, and the host cell to be transformed. A vector contemplated by the present invention is at least capable of directing the replication or insertion into the host chromosome, and preferably also expression, of the structural gene included in the rDNA molecule.

Expression control elements that are used for regulating the expression of an operably linked protein encoding sequence are known in the art and include, but are not limited to, inducible promoters, constitutive promoters, secretion signals, and other regulatory elements. Preferably, the inducible promoter is readily controlled, such as being responsive to a nutrient in the host cell's medium.

In one embodiment, the vector containing a coding nucleic acid molecule will include a prokaryotic replicon, i.e., a DNA sequence having the ability to direct autonomous replication and maintenance of the recombinant DNA molecule extrachromosomally in a prokaryotic host cell, such as a bacterial host cell, transformed therewith. Such replicons are well known in the art. In addition, vectors that include a prokaryotic replicon may also include a gene, whose expression confers a detectable marker such as a drug resistance. Typical bacterial drug resistance genes are those that confer resistance to ampicillin or tetracycline.

Vectors that include a prokaryotic replicon can further include a prokaryotic or bacteriophage promoter capable of directing the expression (transcription and translation) of the coding gene sequences in a bacterial host cell, such as E. coli. A promoter is an expression control element fonned by a DNA sequence that permits binding of RNA polymerase and transcription to occur. Promoter sequences compatible with bacterial hosts are typically provided in plasmid vectors containing convenient restriction sites for insertion of a DNA segment of the present invention. Typical of such vector plasmids are pUC8, pUC9, pBR322 and pBR329 available from Biorad Laboratories, (Richmond, CA), pPL and pKK223 (Pharmacia; Piscataway, N.J.).

Expression vectors compatible with eukaryotic cells, preferably those compatible with vertebrate cells, can also be used to form a rDNA molecules that contains a coding sequence. Eukaryotic cell expression vectors are well known in the art and are available from several commercial sources. Typically, such vectors are provided containing convenient restriction sites for insertion of the desired DNA segment. Typical of such vectors are pSVL and pKSV-10 (Pharmacia), pBPV-1/pML2d (International Biotechnologies, Inc.), and pTDTI (ATCC, #31255), and the like eukaryotic expression vectors.

Eukaryotic cell expression vectors used to construct the rDNA molecules of the present invention may further include a selectable marker that is effective in an eukaryotic cell, preferably a drug resistance selection marker. A preferred drug resistance marker is the gene whose expression results in neomycin resistance, i.e., the neomycin phosphotransferase (neo) gene (Southern et al., J. Mol. Anal. Genet. 1: 327-41 (1982)). Alternatively, the selectable marker can be present on a separate plasmid, and the two vectors are introduced by co-transfection of the host cell, and selected by culturing cells with the appropriate drug for the selectable marker.

The present invention further provides host cells transformed with a nucleic acid molecule that encodes a protein of the present invention. The host cell can be either prokaryotic or eukaryotic. Eukaryotic cells useful for expression of a protein of the invention are not limited, so long as the cell line is compatible with cell culture methods and compatible with the propagation of the expression vector and expression of the gene product. Preferred eukaryotic host cells include, but are not limited to, yeast, insect and mammalian cells, preferably vertebrate cells such as those from a mouse, rat, monkey or human cell line. Preferred eukaryotic host cells include Chinese hamster ovary (CHO) cells (ATCC No. CCL61), NIH Swiss mouse embryo cells NIH/3T3 (ATCC No. CRL 1658), baby hamster kidney cells (BHK), fibroblasts and similar eukaryotic tissue culture cell lines.

Any prokaryotic host can be used to express a rDNA molecule encoding a protein of the invention. A preferred prokaryotic host is E. coli.

Transformation or transfection of appropriate cell hosts with a rDNA molecule of the present invention is accomplished by well known methods that typically depend on the type of vector used and host system employed. With regard to transformation of prokaryotic host cells, electroporation and salt treatment methods are typically used, see, for example, Cohen et al., Proc. Natl. Acad. Sci. USA 69: 2110, (1972); and MANIATIS et al., MOLECULAR CLONING, A LABORATORY MANUAL, Cold Spring Harbor Laboratory, Cold Spring Harbor, N.Y. (1982) and SAMBROOK ET AL., (1989). With regard to transformation of vertebrate cells with vectors containing rDNAs, electroporation, cationic lipid or salt treatment methods are typically utilized, see, for example, Graham et al., Virol. 52: 456-67 (1973); Wigler et al., Proc. Natl. Acad. Sci. USA 76: 1373-76 (1979).

Successfully transformed cells, i.e., cells that contain a new nucleic acid molecule (e.g., rDNA) of the present invention, can be identified by well known techniques including the selection for a selectable marker. For example, cells resulting from the introduction of an rDNA of the present invention can be cloned to produce single colonies. Cells from those colonies can be harvested, lysed and their DNA or RNA content examined for the presence of an oscillogenin nucleic acid using a method such as that described by Southern, J. Mol. Biol. 98: 503-17 (1975) or Berent et al., Biotech. 3: 208 (1985) or the proteins produced from the cell assayed via a suitable immunological detection method.

Recomibinant Oscillogenin Protein. The present invention further provides methods for producing a protein of the invention using recombinant nucleic acid molecules herein described. In general terms, the production of a recombinant form of a protein typically involves the following steps:

First, a nucleic acid molecule is obtained that encodes oscillogenin protein of the invention. The coding sequence, preferably lacking introns, is directly suitable for expression in any host. The sequence can be transfected into host cells, such as eukaryotic cells or prokaryotic cells. Eukaryotic hosts include mammalian cells, as well as insect cells (e.g, Sf9 cells) using recombinant baculovirus. Alternatively, fragments encoding only portion of oscillogenin can be expressed alone or in the form of a fusion protein. The fusion proteins can be purified and used to generate polyclonal antibodies.

The nucleic acid molecule is then preferably placed in operable linkage with suitable control sequences, as described above, to form an expression unit containing open reading frame (ORF) of oscillogenin. The expression unit is used to transform a suitable host, and the transformed host is cultured under conditions that allow the production of the recombinant protein. Optionally, the recombinant protein is isolated from the medium or from the cells. Recovery and purification of the protein may not be necessary in some instances where some impurities may be tolerated.

Each of the foregoing steps can be done in a variety of ways. For example, the desired coding sequences may be obtained from genomic fragmnents and used directly in appropriate hosts. The construction of expression vectors that are operable in a variety of hosts is accomplished using appropriate replicons and control sequences, as set forth above. The control sequences, expression vectors, and transformation methods are dependent on the type of host cell used to express the gene. Suitable restriction sites can, if not normally available, be added to the ends of the coding sequence so as to provide an excisable gene to insert into these vectors. A skilled artisan can readily adapt any host/expression system known in the art for use with the nucleic acid molecules of the invention to produce the desired recombinant protein or polypeptide.

IV. Method of Parthenoienetically Activating Oocvtes Using Oscillogenin for Nuclear Transfer

An important embodiment of the invention is directed to use of oscillogenin for parthenogenetic activation of oocytes. Such activation can occur by (1) administering oscillogenin alone or in combination with other Ca²⁺ oscillating agents, and (2) administering oscillogenin in combination with a sperm or a somatic cell. Oscillogenin can be used in combination with sperm for intracytoplasmic sperm injection (ICSI), sperm fertility testing (e.g., efficacy of oscillogenin augmentation), and for in vitro fertilization (IVF).

In vitro fertilization procedures. Fertilization procedures can be used as described as in Long et al., Mol. Reprod. Dev. 36:23-32 (1993); Alan O. Trounson et a!., HANDBOOK OF IN VITRO FERTILIZATION (1999); and Brigid Hogan et al., MANIPULATING THE MOUSE EMBRYO: A LABORATORY MANUAL (Cold Spring Harbor Laboratory, 1994). Typically, for example, pooled semen, which even can be cryopreserved, is processed using the Percoll method (Hossain et al., Arch. Androl. 37: 189-95 (1996)). The separated motile sperm are added at a final concentration of 500,000 sperm/ml. Heparin (10 μg/ml; Sigma) is added to the fertilization medium to induce sperm capacitation (Parrish et al., Biol. Reprod. 38: 1171-80 (1988)). Eggs are incubated with sperm for at least 4 hours before monitoring. Eggs that subsequently exhibit [Ca²⁺]ᵢ oscillations are fixed and stained to confirm fertilization. The fixation and staining procedures and the criteria used to classify the fertilization stages of a zygote are as described by Fissore et al., Biol. Reprod. 47: 960-9 (1992) and Long et al., (1993).

Medium, Calcium Ionophores, Phosphatases and Protein Kinase Inhibitors. In addition to injecting oscillogenin into an enucleated oocyte or a nucleated oocyte, microinjected oocytes, nuclei from another cell, the cells also can be incubated in a medium enriched with calcium ions (Ca²⁺). Alternatively, or in addition to culturing in Ca²⁺ enriched medium, the oocyte can be coinjected with or exposed to calcium ionophores (e.g., ionomycin and A23187), protein kinase inhibitors (e.g., 6-dimethylaminopurine (DMAP), butyrolactone, roscovitine, p34(cdc2) inhibitors, staurosporine, 2-aminopurine and sphingosine or other serine-threonine kinase inhibitors) or phosphatases (e.g., phosphatase 2A or phosphatase 2B) to enhance the calcium oscillations in the cell (e.g., oocyte). Incubation in calcium ion enriched mediums can be carried out as described by Wang et al., Mol. Reprod. Dev. 53: 99-107 (1999). Also, other divalent cations can be utilized to activate at least rodent oocytes, such as magnesium, strontium, and barium. Divalent cation levels can also be increased using electric shock, oocyte treatment with ethanol and treatment of oocytes with caged chelators.

Calcium ionophores are typically used in combination with protein kinase inhibitors. Embodiments of this invention contemplate use of either an ionophore and oscillogenin or a protein kinase inhibitor and oscillogenin, or all three. Protein kinase inhibitors can be utilized as described in U.S. Pat. No. 5,945,577. Calcium ionophores, in combination with protein kinase inhibitors, can be used as described in Susko-Parrish et al., Dev. Biol. 166: 729-39 (1994); Mitalipov et al., Biol. Reprod. 60: 821-7 (1999); Liu et al., Biol Reprod. 61: 1-7 (1999); Mayes et al., Biol. Reprod. 53: 270-5 (1995); and U.S. Pat. No. 5,496,720. Typically, oocytes are briefly (e.g., approximately 5 minutes) exposed to the ionophore. Phosphatases also can be used to increase calcium levels as described in U.S. Pat. No. 5,945,577.

Parthenogenetic Activation of Oocytes. Parthenogenetic activation of oocytes can be induced several ways including: (1) basic treatment with a Ca-ionophore and cytochalasin D combined with cycloheximide; (2) electric impulse; (3) cycloheximide and electric pulse treatments (see Bodo et al., Acta Vet. Hung 46: 493-500 (1998)); (4) combined use of calcium ionophores (e.g., A23187) and protein kinase C stimulators (e.g., phorbol esters) (Uranga et al., Int'l. J. Dev. Biol. 40: 515-9 (1996)); (5) oocyte exposure to 7% (v/v) ethanol solution (Lai et al., Reprod. Fertil. Dev. 6: 771-5 (1994)); (6) induction using puromycin (De Sutter et al., J. Assist. Reprod. Genet. 9: 328-37 (1992)); (7) incubation of oocytes in strontium ion enriched medium (O'Neill et al., Mol. Reprod. Dev. 30: 214-9 (1991)); and (8) 200 μm thimerosol, which has been observed to induce Ca⁺² oscillation in pig oocytes (Machaty et al., Biol. Reprod. 57: 1123-7 (1997)). In addition to methods of inducing parthenogenetic activation, the efficacy of activation can be affected by cryopreservation of the oocytes (see, e.g., Lai et al., Reprod. Fert. Dev. 6: 771-5 (1994)). Consequently, another embodiment of this invention is to compensate for lower parthenogenetic efficiencies induced by cryopreservation, as well as to provide new materials of improving overall efficacy of parthenogenetic activation of oocytes using freshly harvested cells.

V. Method of Enhancing ICSI

Another embodiment of the application is to use oscillogenin to enhance the ICSI efficacy in both animal husbandry and in vitro fertilization (IVF). Oscillogenin can be used alone with the ICSI technique, or in combination with one or more calcium ionophores, protein kinase inhibitors, phosphatases or calcium enriched mediums, as discussed above. To further enhance sperm-oocyte fusion, electrical stimulation can be utilized as described by Yanagida et al., Hum. Reprod. 14: 1307-11 (1999)).

Another method which can be used in combination with those listed above is the vigorous aspiration of oocyte cytoplasm to improve ICSI outcomes as described by Tesarik et al., Fertil. Steril. 64: 770-6 (1995).

Kinase Assays. Kinase assays can be used to determine if oscillogenin-induced [Ca²⁺]ᵢ oscillations are capable of evoking oocyte activation, and thus determine the efficiency of each of the above combinations of techniques and compositions. Suitable kinase assays include histone H1 and mitogen-activated protein (MAP) kinase assays, which can be performed as described by Fissore et al., Biol. Reprod. 55: 1261-70 (1996). Myelin basic protein (MBP) is assumed to measure mostly MAP kinase activity, as shown previously (Id.). Groups of five eggs are transferred into 5 μL of an H1 kinase buffer solution containing 10 μg/ml aprotinin, 10 μg/ml leupeptin, 10 μg/ml pepstatin A, 500 nM protein kinase A inhibitor, 80 mM β-glycerophosphate, 20 mM EGTA, 15 mM MgCl, and 1 mM dithiothreitol (DTT) (as described by Collas et al., Mol. Reprod. Dev. 34: 224-231 (1993)). Eggs are lysed with repeated cycles of freezing and thawing and stored at −80° C. until the kinase assay is performed.

Kinase reactions are started by adding 5 μl of a solution containing 2 mg/ml histone H1 (type III-S, Sigma), 1 mg/ml MBP (Sigma), 0.7 mM ATP, and 50 μCi of [γ³²P] (Amersham, Arlington Heights, Ill.) to 5 μl of the crude egg lysates. The reaction is carried out for 30 min. at 30° C. and terminated by the addition of 5 μl of SDS sample buffer (Laemmli, Nature 227: 680-685 (1970)). Samples are boiled for 3 min. and loaded onto about a 12 or 15% SDS-polyacrylamide gel. Control samples typically contain all the components for the reaction except oocytes. Phosphorylation of histone H1 and MBP is visualized by autoradiography using DuPont's Cronex intensifying screens at −70° C. or other similar system. Such kinase assays can be used to assess sperm specimen fertility, as well as assess the efficacy of a specific combination of techniques and/or compositions. Other conditions for performing the kinase assay would be known to the skilled artisan.

Additional methods to determine whether the oocyte has been induced into a pathway of fertilization, at least in rodents, can be determined by whether the second polar body is extruded. Extrusion of the second polar body can be visualized via microscopy. Also, down-regulation of the inositol triphosphate receptor (IP₃R) only appears to occur following fertilization, SF injection and inositol triphosphate (IP₃) injection, but not when oocytes are exposed to ethanol, calcium ionophores or strontium chloride. Down-regulation of IP₃R can be assessed both at the level of RNA transcription or at protein synthesis. Such methods are commonly known in the art, for example see ED HARLOW ET AL., ANTIBODIES: A LABORATORY MANUAL (1988); and SAMBROOK ET AL., CLONING: A LABORATORY MANUAL (1989).

VI. Method and Kit for Assessing Sperm Fertility Another embodiment of the invention is to measure the oscillogenin content of sperm as a means of measuring sperm fertility. The content can be measured by detecting the concentration and/or localization of oscillogenin in sperm. Oscillogenin can be assessed using antibodies or immunogenic fragments thereof that recognize and bind to oscillogenin. Alternatively, oscillogenin can also be assessed using nucleic acid probes that detect mRNAs. These procedures can be performed using any of the following techniques or as described in the examples.

In one embodiment, the present invention provides a method for determining fertility, by measuring the presence and concentration of oscillogenin in the sperm of the animal being tested. In this method the presence or absence of oscillogenin in a sperm sample is assayed by measuring the amount, if any, of oscillogenin in the sperm from the sample which binds to an anti-oscillogenin antibody. The anti-oscillogenin antibody can be polyclonal, but is preferably monoclonal. Oscillogenin can also be identified the monoclonal antibody of the present invention.

Typically in domesticated animals, there are about 1×10⁹ sperm/ml of ejaculate. Fertility of an animal can then be determined by screening the collected sample for the presence and amount of oscillogenin. Testing using antibodies can be performed using Western blot assays, ELISAs and other immune assays as would be known to the skilled artisan.

Enzymiie Linked Immtunosorbent Assay (ELISA). A preferred immunologic means of detecting oscillogenin is the ELISA method. A protein sample is then contacted with these plates. The samples are preferably prepared by diluting oscillogenin removed from a known number of sperm in an incubation-suitable buffer. The samples are placed in the well, incubated at a temperature ranging from about 25° C. to about 37° C., and preferably at about 37° C. for a time period of from about 1 hour to about 4 hours, and preferably about one hour. The wells containing the sample are washed thoroughly before introducing a detection antibody (e.g., anti-oscillogenin antibodies) into the well.

An antibody can be directly labeled or detected using a second antibody. The label may suitably be any which is conventionally attached to monoclonal antibodies or antibody fragments for use in an immunoassay, such as an enzyme (e.g., horseradish peroxidase), a chromophore, a fluorophore (e.g., green fluorescent protein, blue fluorescent protein, or luciferase), or a radiolabel (e.g., ¹²⁵I). The label may be bonded to the monoclonal antibody by any conventional method including via conventional cross-linking agents. See ED HARLOW ET AL., ANTIBODIES: A LABORATORY MANUAL (1988).

Western Blot and Immunoprecipitation. For assessing steady-state protein concentrations, Western blots can be used. For Western blots, a typical procedure can be performed as follows. Equal number of spermatozoa contained in, for example, 200 μl of semen is added to a 1.5 ml microcentrifuge tube with 1 ml phosphate buffered saline (PBS) containing TWEEN and 1% bovine serum albumin (BSA) and protease inhibitors, and centrifuged at 4000 rpm to remove seminal fluid. The sperm can be washed 2-3 times before adding sample buffer (Laemmli, 1970) and boiling for 5 min prior to being applied to either 10-15%, preferably 12%, polyacrylamide gels, transferred and Western blotted.

For immunoprecipitation, a typical procedure can be performed by conjugating a monoclonal antibody to HZ Beads (Sigma Chemical Co., St. Louis, Mo.) or similar beads. The membranes of washed sperm are lysed with detergent or with mechanical means and then removed by centrifugation. The beads are added to the supernatant and protein is allowed to bind to the antibody (˜10 min). The beads are then washed three times, boiled in sample buffer and the sample buffer is applied to 10-15%, preferably 12%, PAGE. The presence of protein can be determined directly using any suitable protein assaying technique such as Coomassie blue staining of the gels, ELISA or by Western blot. Other methods of immunodetection are as described in HARLOW ET AL., (1988).

Inmunofluorescence. Antibodies which recognize and bind to oscillogenin can be used in conjunction with secondary antibodies with fluorescent tags. The fluorescent tags can be fluorescein, rhodamine, rhodamine GREENÔ and other like fluorescent labels.

Electron microscopic analysis. In another embodiment of the invention, electron microscopy can be used to assess the concentration and location of oscillogenin in spermatozoa. Spermatozoa can be fixed for electron microscopy by the procedure described by R. C. Jones, J. Reprod. Fertil. 193: 145-149 (1973).

VII. Method and Compositions for Modulating Oscillopenin Activity

An embodiment of the invention involves compositions and methods of modulating oscillogenin activity and thereby sperm fertility and/or oocyte activation.

For example, oscillogenin can be administered into a targeted oocyte either alone or in combination with (1) a sperm or its genetic material or (2) a somatic cell or its genetic material. When oscillogenin is administered in combination with for example, a sperm, oscillogenin can be administered prior to, simultaneously with, or immediately after injection of, for example, a sperm. Oscillogenin can further be administered with any of the agents which regulate calcium ion oscillations.

VIII. Antibodies Another embodiment of the invention is antibodies or immunogenic fragments which recognize and bind to oscillogenin. Anti-oscillogenin antibodies are prepared by immunizing suitable mammalian hosts using appropriate immunization protocols and oscillogenin or immunogenic peptides thereof. These peptides can be at least 5, 6, 7, 8, 9, 10, 15, 20, 25, 30, 40 or 50 consecutive amino acids in length, or the entire oscillogenin protein. Oscillogenin or an immunogenic fragment thereof, may be conjugated to suitable carriers. Methods for preparing immunogenic conjugates with carriers such as bovine serum albumin (BSA), keyhole limpet hemocyanin (KLH), or other carrier proteins are well known in the art. In some circumstances, direct conjugation using, for example, carbodiimide reagents may be effective; in other instances linking reagents such as those supplied by Pierce Chemical Co., Rockford, Ill., may be desirable to provide accessibility to the hapten. The hapten peptides (such as the lengths described above) can be extended at either the amino or carboxy terminus with a Cys residue or interspersed with cysteine residues, for example, to facilitate linking to a carrier.

Administration of the immunogens is conducted generally by injection over a suitable time period and with use of suitable adjuvants, as is generally understood in the art. During the immunization schedule, titers of antibodies are taken to determine adequacy of antibody formation.

Anti-peptide antibodies can be generated using synthetic peptides corresponding to, for example, the amino or carboxy terminal 15-20 amino acids of oscillogenin. Synthetic peptides can be as small as 2-3 amino acids in length, but are preferably at least about 4 to about 20 or more amino acid residues long. The peptides are coupled to KLH using standard methods and can be immunized into animals such as rabbits. Other animals such as rodents (e.g., mice), sheep, goats, horses and other ungulates may also be used. Polyclonal anti-oscillogenin antibodies or peptide antibodies can then be purified, for example using Actigel beads containing the covalently bound peptide or protein.

While the polyclonal antisera produced in this way may be satisfactory for some applications, for pharmaceutical compositions, use of monoclonal (InAb) preparations is preferred. Immortalized cell lines, which secrete the desired monoclonal antibodies, may be prepared using the standard method of Kohler and Milstein (Nature 256: 495-7 (1975)) or modifications which effect immortalization of lymphocytes or spleen cells, as is generally known (see, e.g., Harlow et al. 1988). The immortalized cell lines secreting the desired antibodies are screened by immunoassay in which the antigen is the peptide hapten, polypeptide or protein. When the appropriate immortalized cell culture secreting the desired antibody is identified, the cells can be cultured either in vitro or by production in ascites fluid.

The desired monoclonal antibodies are then recovered from the culture supernatant or from the ascites supernatant. Fragments of the monoclonal or the polyclonal antisera, which contain the segment which recognizes and binds to oscillogenin, can be used to label oscillogenin as well as potential regulators of oscillogenin. Use of immunologically reactive fragments, such as the Fab, SCFV, Fab′, of F(ab′)₂ fragments is often preferable, especially in a therapeutic context, as these fragments are generally less immunogenic than the whole immunoglobulin.

### EXAMPLES

### Example 1

### Method of Enriching Oscillogenin

The following procedure can be used to enrich the sperm factor (SF), a Ca²⁺-release activating protein, by sequential chromatography and to identify the effector polypeptide by comparative SDS-polyacrylamide gel electrophoresis (PAGE).

To fractionate boar sperm factor (SF) by sequential chromatography and to identify the candidate polypeptide the procedures are:
a) Precipitation with ammonium sulfate. The active material precipitates at 50% saturated solution and provides a 2-fold enrichment of the [Ca²⁺]ᵢ-releasing activity.
b) Affinity chromatography on HiTrap blue dye. The active polypetide elutes as peak no. 3 with 1 M KCl. This gives an enrichment of 4-fold.
c) Hydroxyapatite chromatography. Elution from this column is accomplished with increasing concentration of phosphate buffer. The active protein elutes with peak no. 3 (185 mM potassium phosphate) and provides a 4-fold enrichment of activity.
d) Size exclusion chromatography on Superose 12. The active protein elutes with peak 4 and has an MW of 43 kDa. This gives an enrichment factor of 5-fold.
   If each of these procedures works independently, the predicted enrichment when used in combination is approximately 128-fold. As the four procedures were based on distinct biochemical properties, it is highly likely that each depletes a distinct set of boar SF proteins. The 50% ammonium sulfate pellets were accumulated from 12 separate boar ejaculates. The equivalent of one boar ejaculate was solubilized and processed through the blue affinity column. The peak no. 3 proteins were collected and stored at −80° C., which fully preserves activity. This process was repeated 12 times. The peak no. 3 fractions from the 12 runs were then combined and loaded onto the hydroxyapatite column. Then peak no. 3 from the hydroxyapatite column was collected, concentrated and immediately poured onto Superose 12. Each individual fraction (250 μl) in the active peak (peak no. 4) of this column were separately concentrated and tested for [Ca²⁺]ᵢ releasing activity.

Our results show that several proteins within fraction 4-2, and which exhibit a MW in between 35-80 kDa, could be involved in the ability of SF to trigger Ca²⁺ release.

Amnimonium sulfate precipitation. Crude sperm extracts were mixed with saturated ammonium sulfate to 50% saturation. The precipitates were collected by centrifugation (10,000×g, 15 min., at 4° C.) and the pellets were stored at −20° C. until used. Pellets were resuspended in injection buffer (75 mM KCl and 20 mM HEPES, pH=7.0), washed in the same buffer, and concentrated using Centricon-30 ultrafiltration membranes before assaying for Ca²⁺ releasing activity.

Chromatography. Columns (all from Phamacia; Pitscataway, N.J.) used in the isolation procedures with fast protein liquid chromatography (FPLC) were utilized according to Reduth et al., J. Eukaryot. Microbiol. 41: 95-103 (1994); Morgan et al., Molec. Biochem. Parasitol 57: 241-52 (1993); Muranjan et al., Infect. Immun. 65: 3806-14 (1997); Wu et al., Dev Biol. 203: 369-81 (1998)). The pumps and tubing that serve the chromatographic system were flushed with absolute ethanol and subsequently with sterile PBS prior to all fractionation. Columns were sterile and are stored with azide to prevent contamination. All buffers were autoclaved and filtered before use. Collection tubes were sterile and coated with silicon to reduce non-specific loss of protein. The FPLC, fraction collector and all buffers were housed in a 4° C. room to further reduce bacterial growth and enzyme activity.

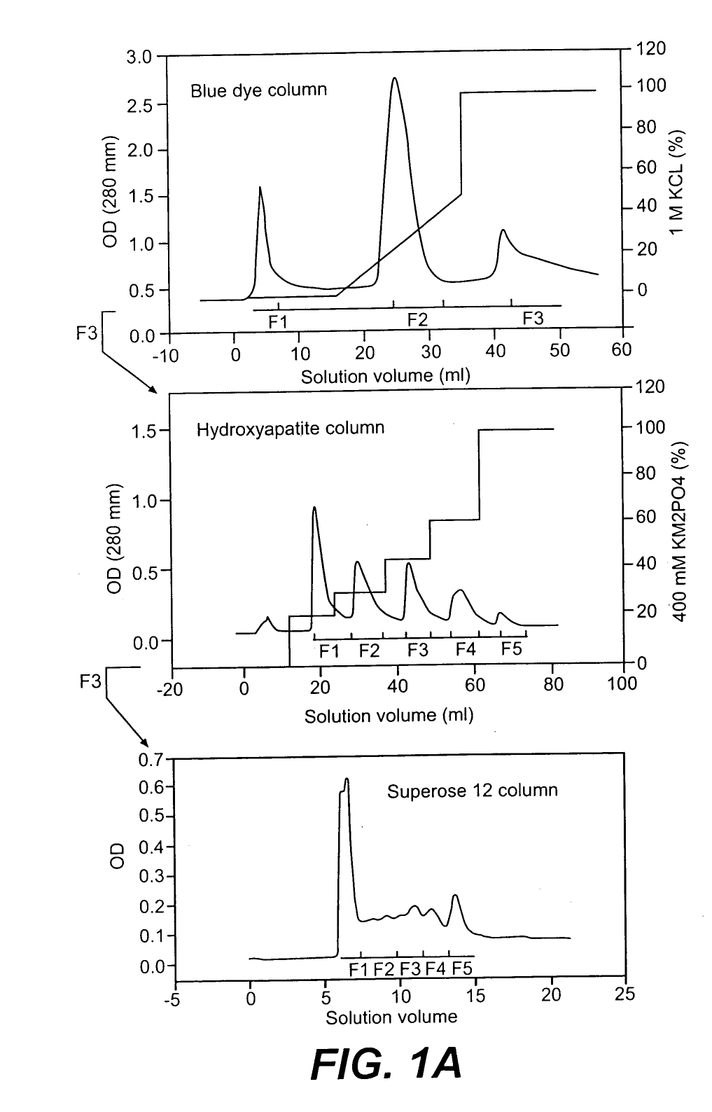

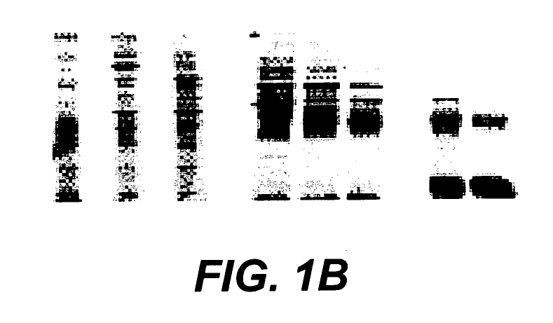

HiTrap blue affinity FPLC chromatography. Anrnonium sulfate pellets were diluted into buffer A (20 mM HEPES, 1 mM EDTA, pH=7.0), and loaded onto a 5 ml HiTrap Blue affinity column (Pharmacia) by using the FPLC system at 4° C. After a 15 ml wash with buffer A, proteins were eluted with a 20-ml linear gradient from 0 to 500 mM KCl, and finally with a 20-ml 1 M KCl. The activity was observed in peak no. 3 (see FIG. 1A). This fraction was concentrated (12 peaks will be accumulated), washed, and poured onto the hydroxyapatite column.

Hydroxyapatite FPLC chromatography. Proteins from peak no. 3 obtained using the HiTrap blue affinity FPLC chromatographic column were diluted into 10 mM potassium phosphate buffer (pH=6.8) with 200 [M phenylmethanesulphonyl fluoride (PMSF) and loaded at 0.4 ml/min onto a 5 ml hydroxyapatite column using FPLC system at 4° C. After 10 ml wash with 10 mM phosphate buffer, proteins were eluted at the same flow rate by increasing the molarity of the potassium phosphate buffer (pH=7.2 with 200 μM PMSF) in a step-wise manner. The potassium phosphate concentration in each step was as follows: 88 mM, 127 mM, 185 mM, 244 mM, 302 mM and 400 mM. Fractions in peak no. 3 (Wu et al., Dev. Biol. 203: 369-81 (1998)) were collected, washed, concentrated and poured onto a Superose 12 column.

Superose 12 FPLC chromatography. The active fractions from hydroxyapatite column (peak 3 concentrated to a total volume less than 250 μl) are loaded at 4° C. onto a Superose 12 HR 10/30 column connected to a FPLC system. Proteins were eluted with buffer (75 mM KCl and 20 mM HEPES, pH=7.0) containing 200 μM PMSF at a flow rate of 0.1 ml/min and detected at OD₂₈₀ by an UV-M monitor. Each individual fraction (0.25 ml) was collected, and concentrated before testing for Ca²⁺ releasing activity. The Superose 12 HR 10/30 column was calibrated using 13-amylase (200 kDa), alcohol debydrogenase (150 kDa), bovine serum albumin (68 kDa) and carbonic anhydrase (29 kDa) (Sigma).

### Example 2

### Characterization of the Oscillogenin Protein

### This Example Characterizes the Activity of an SF Extract

Egg Recovery and Culture

Mouse eggs or recently fertilized zygotes were recovered from the oviducts of 8-20 week old CD-1 female mice as previously described (Wu et al., Dev. Biol. 203, 369-81 (1998)). Mice were superstimulated with an injection of 5 I.U. pregnant mare serum gonadotropin (PMSG; Sigma Chemical Co., St. Louis, Mo.; all reagents from Sigma unless specified), and induced to ovulate 40-48 hr later by injection of 5 I.U. human chorionic gonadotropin (hCG; Sigma). To obtain fertilized zygotes, females were placed overnight with males following the injection of hCG. Eggs were collected 14 hr post-hCG (phCG) injection into a HEPES-buffered solution (TL-Hepes) supplemented with 5% heat-treated fetal calf serum (FCS; Gibco, Grand Island, N.Y.). Granulosa cells were removed by a 5-10 min incubation with bovine testis hyaluronidase, and oocytes showing no signs of degeneration and first polar body extrusion were selected for these studies. Eggs were transferred to 50 μI drops of KSOM (Specialty Media, Phillipsburg, N.J.), where they were incubated before and after activation for variable periods of time under paraffin oil at 36.5° C. in a humidified atmosphere containing 7% CO₂ in air.

Microinjection Techniques

Microinjection procedures were carried out as previously described (Wu et al., Dev. Biol. 203: 369-81 (1998)). Briefly, eggs were placed in a 50 μl microdrop of TL-Hepes supplemented with 2.5% sucrose and 20% FCS under paraffin oil and injected using manipulators (Narishige, Medical Systems Corp., Great Neck, N.Y.) mounted on a Nikon Diaphot microscope (Nikon, Inc., Garden City, N.Y.). Injection pipettes were loaded by suction from a 2-3 μl drop containing one of the following compounds: 0.5 mM fura-2 dextran (fura-2 D; Molecular Probes, Eugene, Oreg.), 1 mg/ml protein concentration of boar sperm fractions (SF), or 10 μM adenophostin A, a powerful IP₃R agonist (courtesy of Dr. K. Tanzawa, Sankyo Colo., Tokyo, Japan). All reagents were diluted in buffer containing 75 mM KCl and 20 mM HEPES, pH 7.0 and were delivered into the ooplasm by pneumatic pressure using a PLI-100 picoinjector (Medical Systems Corp.). Injection volumes were approximately of 5-10 pl, and this resulted in intracellular concentrations of approximately 10 ng/μl for SF (2.5-5 sperm equivalents; Wu et al., 1998), and 100 nM for adenophostin A.

SF Preparation

Cytosolic SF extracts were prepared from boar semen as described by Swann, Development 110: 1295-1302 (1990); and Wu et al, Dev. Biol. 203: 369-81 (1998). In brief, semen samples were first washed twice with TL-Hepes, and the pellet resuspended in a solution containing 75 mM KCl, 20 mM HEPES, 1 mM EGTA, 10 mM glycerophosphate, 1 mM DTT, 200 μM PMSF, 10 μg/ml pepstatin, and 10 μg/ml leupeptin, pH 7.0. The sperm suspension was lysed by sonication (XL2020, Heat Systems Inc., Farmingdale, N.Y.) using a small probe at a setting of 3 for 15-25 min at 4° C. The sonicated suspension was spun twice at 10,000 ×g, the supernatant collected both times, and then centrifuged at 100,000×g for 45 min at 4° C. The resulting clear supernatant was collected as the cytosolic fraction. Ultrafiltration membranes (Centricon-50, Amicon, Beverly, Mass.) were used to wash the supernatants (75 mM KCl and 20 mM HEPES, pH 7.0). The extracts were then precipitated by exposure to a saturated solution of ammonium sulfate (50% final concentration), followed by centrifugation at 10,000 g for 15 min at 4° C. The precipitates were collected and stored at −80° C. until the time of use.

Parthenogenetic Activation

Several commonly used parthenogenetic agents were used during the course of these studies, including ethanol and ionomycin, which induce a single [Ca²⁺]ᵢ rise (Cuthbertson, J. Exp. Zool. 226: 311-14 (1983); and Shiina et al., J. Reprod. Fert 97: 143-50 (1993)), and others such as adenophostin A, SF, thimerosal, and SrCl₂ that evoke [Ca²⁺]ᵢ oscillations (Kline et al., Dev. Biol. 149: 80-9 (1992); Swann, Biochem J. 287: 79-84 (1992); and Sato et al., Biol. Reprod. 58: 867-73 (1998)). The activating compounds were either injected into eggs (e.g., adenophostin A and SF, see microinjection procedures for details) or added to the eggs' culture media (e.g., thimerosal, SrCl₂). Activation was started in all cases at 16 hr post hCG administration (phCG) and eggs evaluated visually 2 hr later by observing second polar body extrusion (18 hr phCG) and 5 hr post-treatment by evaluation of pronuclear formation. After the activation procedure and the assigned incubation period (1, 2, 4 or 8 hr), eggs were collected in 5 μl Dulbecco's phosphate buffered solution (DPBS)/polyvinylpyrrolidone (3 mg/ml, PVP) and stored at −80° C. The great majority of eggs collected 8 hr post-treatment, except those treated with thimerosal, exhibited pronuclear formation.

Ethanol activation was carried out by exposing eggs to a 7% ethanol solution in TL-Hepes plus 3 mg/ml bovine serum albumin (BSA) for 5 min at 37° C. After the treatment, eggs were washed several times in TL-Hepes, and cultured for 8 hr (24 hr-phCG). Activation with ionomycin and 6-dimethylaminopurine (DMAP), a kinase inhibitor (Susko-Parrish et al., Dev. Biol. 166: 729-39 (1994)) was accomplished by incubating eggs with 5 μM ionomycin for 5 min in Ca²⁺ free-DPBS plus 3 mg/ml BSA. Eggs were then washed in TL-Hepes/1 mg/ml BSA, placed in DMAP/KSOM (2 mM) for 4 hr and cultured for 4 hr after carefuilly washing them free of DMAP. Eggs activated with SrCl₂ were incubated for 2 or 4 hr in a Ca²⁺-free M-16-like medium supplemented with 10 mM SrCl₂. The treated eggs were then washed in TL-Hepes supplemented with 5% FCS and cultured for 2, 4 or 6 hr, depending on the experiment. For thimerosal activation, eggs were exposed to freshly made solutions of 200 μM thimerosal in KSOM for 30 min. Thimerosal-treated eggs were washed free of the reagent using TL-Hepes supplemented with 5% FCS and incubated for 30 min, 1.5, 3.5 or 7.5 hr.

Fluorescence Recordings and [Ca²⁺]ᵢ Determinations Monitoring of [Ca²⁺]ᵢ levels using fura-2 D loaded eggs was carried out as previously described (Wu et al., 1998). UV illumination was provided by a 75 watt xenon arc lamp, and 340 and 380 nm excitation wavelengths were utilized. The intensity of the UV light was attenuated 32-fold with neutral density filters, and a photomultiplier tube quantified the emitted light after passing through a 500 nm barrier filter. The fluorescence signal was averaged for the whole egg. A modified Phoscan 3.0 software program run on a 486 IBM-compatible system controlled the rotation of the filter wheel and shutter apparatus to alternate wavelengths. Free [Ca²⁺]ᵢ was determined from the 340 nm/380 nm ratio of fluorescence. Rmin and Rmax were calculated using 10 pM fura-2 D in Ca²⁺-free DPBS supplemented with 2 mM EDTA (Rₘᵢₙ) or 2 mM CaCl₂ (Rₘₐₓ) and with 60% sucrose to correct for intracellular viscosity (Grynkiewicz et al., J. Biol. Chem. 260: 3440-50 (1985); and Poenie, Cell Calcium 11: 85-91 (1990)). The same solutions were also used alone for background subtractions. Ca²⁺ measurements carried out in the presence of extracellular SrCl₂ are presented as fluorescence ratios of the 340 nm/380 nm excitation wavelengths. Calculations of the [Ca²⁺]ᵢ concentration was in this case not performed, since fura-2 has also some affinity for Sr²⁺. The presence of intracellular Sr²⁺ therefore interferes and prevents obtaining accurate Ca²⁺ and/or Sr2+ intracellular concentrations (Hajnoczky et al., EMBO J., 16: 3533-43 (1997)).

Eggs were measured individually in 35 μl drops of TL-Hepes medium placed on a glass coverslip on the bottom of a plastic culture dish under paraffin oil. Fluorescence ratios were measured every 6 sec, and readings were taken for 1 sec at each wavelength. Oocytes were first monitored for 10-120 sec to establish baseline [Ca²⁺]ᵢ values, after which, recordings were stopped for 2-6 min to allow for microinjection or addition of reagents. Recordings were then restarted and continued for 10-30 min.

Inhibitor Preparation

Lactacystin (100 μM; Calbiochem, La Jolla, Calif.), a proteasome inhibitor (Mellgren, J. Biol. Chem. 272: 29899-903 (1997); and Fenteany et al., J. Biol. Chem. 273: 8545-48 (1998)), was used to determine if the proteasome was involved in down-regulation of the IP₃R-1 by SF. Eggs were incubated in the inhibitor for 30 min. prior to injections, and injections of SF were carried out in the presence of the inhibitor. Eggs were then cultured in a new drop of KSOM freshly supplemented with lactacystin for 2 hr.

Western Blot Technique

Equal volumes of crude lysates from 15 or 20 mouse eggs and double strength sample buffer (Laemmli, Nature 227: 680-5 (1970)) were combined as previously described (He et al., Biol. Reprod. 57: 1245-55 (1997)). Samples were boiled for 3 min and loaded into a 4% SDS-polyacrylamide gels. The separated proteins were transferred onto nitrocellulose membranes (Micron Separation; Westboro, Mass.) using a Mini Trans Blot Cell (Bio-Rad; Hercules, Calif.) for 2 hr at 4° C. The membranes were first washed in PBS and 0.05% Tween (PBS-T) and then blocked in 6% nonfat dry milk in PBS-T for 1 hr. After several washes in PBS-T, the membranes were incubated overnight with a rabbit polyclonal antibody raised against a 15 peptide sequence of the C-terninal end of the IP₃R-1 subtype (Rbt04) diluted to 1:3,000 in PBS-T (Parys et al. Cell Calcium, 17: 239-49 (1995)). Following several washes, the membranes were incubated for 1 hr with a secondary antibody coupled to horseradish peroxidase and diluted 1:3,000 in PBS-T. The membranes were developed using western blot chemiluminescence reagents (NEN Life Science Products; Boston, Mass.) and exposed for 1-3 min. to maximum sensitivity film (Kodak, Fisher Scientific; Springfield, N.J.). Broad range, pre-stained SDS-PAGE molecular weight markers (Bio-Rad) were run in parallel to estimate the molecular weight of the immunoreactive bands. The intensity of the IP₃R-1 bands was quantified using Adobe Photoshop (Mountain View, Calif.) essentially as described by Cameron et al., Cell 83: 463-72 (1995) and plotted using Sigma plot software (Jandel Scientific Software; San Rafael, Calif.). The mean pixel intensity within a selected set area containing each IP₃R-1 band was obtained, and the same set area was applied to all lanes for that particular film. The same set area was also placed in an area of the film where there were no bands, and a background number was taken. This background number was then subtracted from all IP₃R-1 densities for the film under consideration. The band from metaphase II (MII) eggs was used as a reference and assigned the value of 1. The intensity of the IP₃R-1 band from eggs after treatment with several different parthenogenetic agents was calculated relative to 1 and statistically compared. To avoid possible saturation of the quantification system and to be sure that quantification was performed in the linear range, 4 or 5 exposures of each film were obtained and quantified. Underexposed and overexposed exposures were discarded. Western blotting procedures were repeated at least three times, and eggs were collected over several different dates.

Statistical Analysis Statistical comparisons of the intensity of IP₃R-1 bands and of the Ca²⁺ parameters were performed using one-way ANOVA. If differences were observed between groups, comparisons between treatments were achieved by applying the Tukey-Kramer method using JMP IN software (SAS Institute; Cary, N.C.). Significance was at p<0.05.

Results

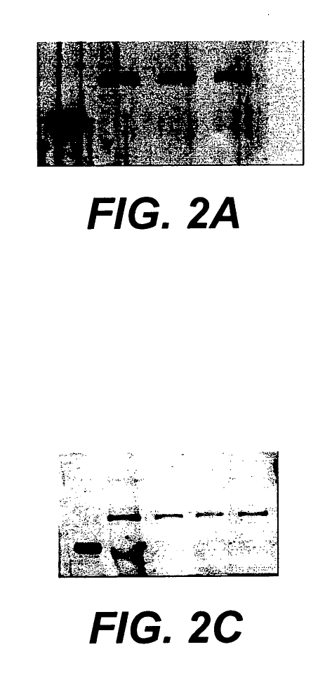

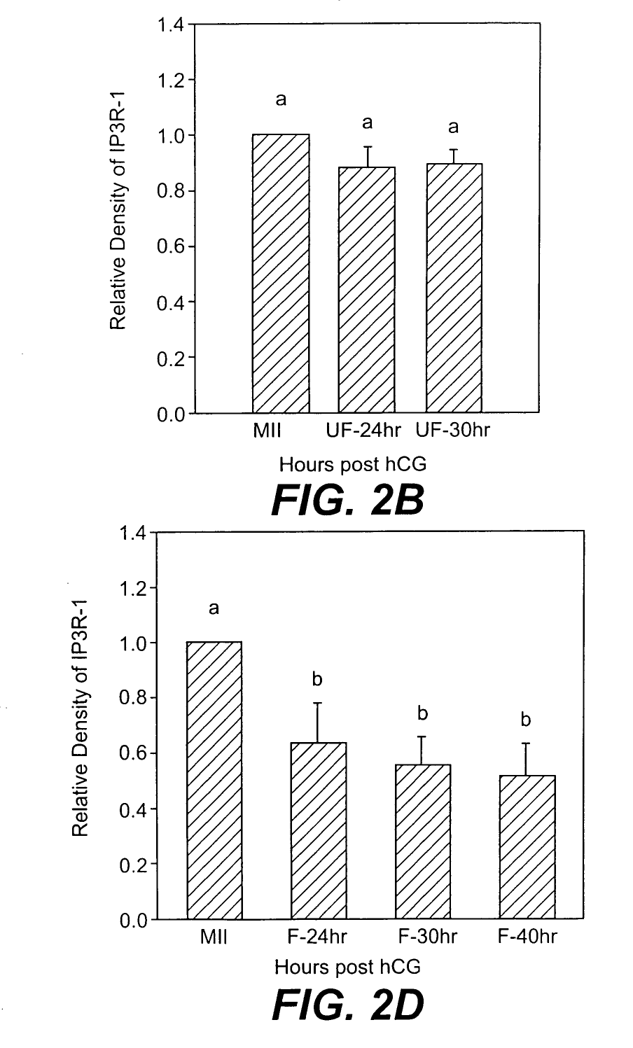

Egg aging andfertilization have dissimilar effects on IP₃R-1 down-regulation Aging of eggs or their fertilization has been shown to induce a marked decrease in the eggs' Ca²⁺ responses to IP₃ injection (Jones et al., Development 121: 3259-66 (1995); and Jones et al., Dev. Biol. 178: 229-37 (1996)). To demonstrate if the reduced responsiveness of the IP₃R1-system was due to a decrease in the number of IP₃R-1, mouse eggs were either collected after ovulation and aged in vitro, or collected soon after fertilization and cultured in vitro for a variable period of time. Unfertilized eggs were cultured for 10 hr (24 hr phCG), or 16 hr (30 hr phCG), and zygotes were cultured to the pronuclear stage (24 hr phCG), to immediately before first mitosis (30 hr phCG), or to the 2-cell stage (40 hr phCG). The presence and amounts of IP₃R-2 in these eggs was assessed by Western blotting. As shown in FIG. 2A, B, aging of eggs in vitro had no significant effect on the amount of IP₃R-1. Conversely, fertilization induced marked down-regulation of the IP₃R-1 by the time of pronuclear formation (FIG. 2C, D). No significant additional down-regulation of the receptor was observed after this time (FIG. 2C, D).

Single [Ca²⁺]ᵢ rises induced by ethanol and ionomycin do not down-regulate IP₃R-1

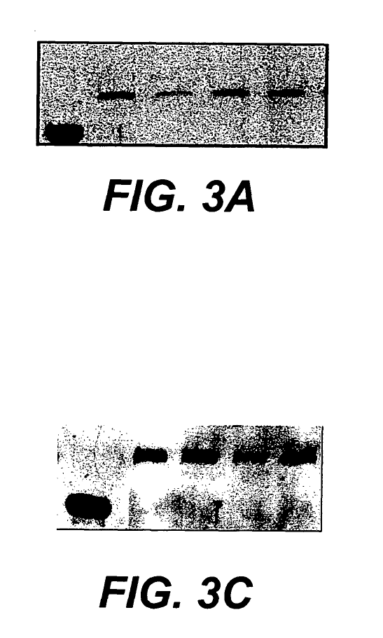

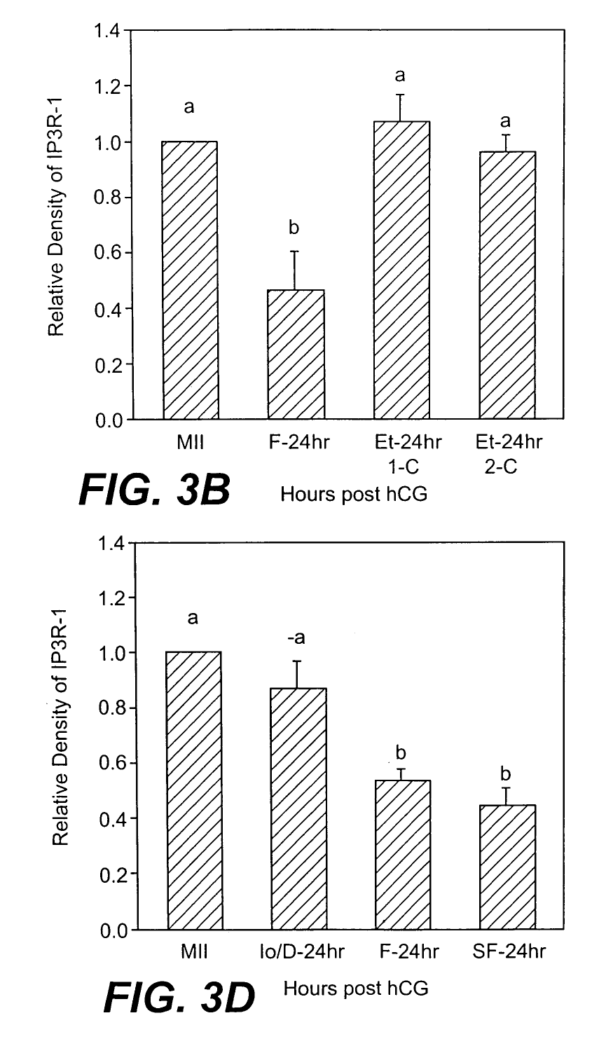

To understand the mechanism(s) by which IP₃R-1 is down-regulated during fertilization, we investigated whether inducing a single [Ca²⁺]ᵢ rise had an impact on IP₃R-1 degradation. It is well established that single Ca²⁺ responses, like those induced by ethanol and ionomycin, trigger high rates of activation and initiation of development in aged oocytes (Cuthbertson, J. Exp. Zool. 226: 311-14 (1983); Shiina et al., J. Reprod. Fert. 97: 143-50 (1993); and Susko-Parrish et al., Dev. Biol. 166: 729-39 (1994). In addition, DMAP, a kinase inhibitor, was added to test the possibility that in the absence of oscillations, additional kinase activity down-regulation may stimulate IP₃R-1 degradation. Thus, we investigated if mouse eggs exposed to 70% ethanol or 5 pM ionomycin+2 mM DMAP and collected at the pronuclear stage (24 hr phCG) exhibited down-regulation of the IP₃R-1. Exposure to these agents was unable to signal IP₃R-1 degradation (FIG. 3), although they induced high rates of activation. However, fertilization and injection of SF induced down-regulation of the receptor (FIG. 3C, D). These results suggest that [Ca²⁺]ᵢ oscillations may be required to induce fertilization-like IP₃R-1 down-regulation.

[Ca²⁺]ᵢ Oscillations Induced by Injection of SF and Adenophostin A, but not by Exposure to SrC]₂, Induce IP₃R-1 Degradation

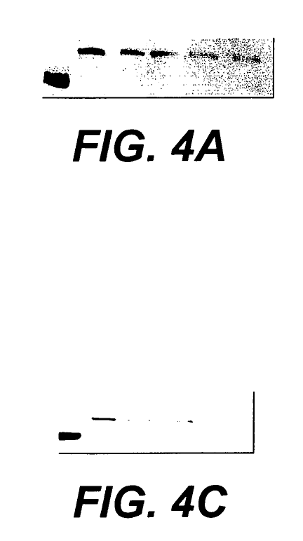

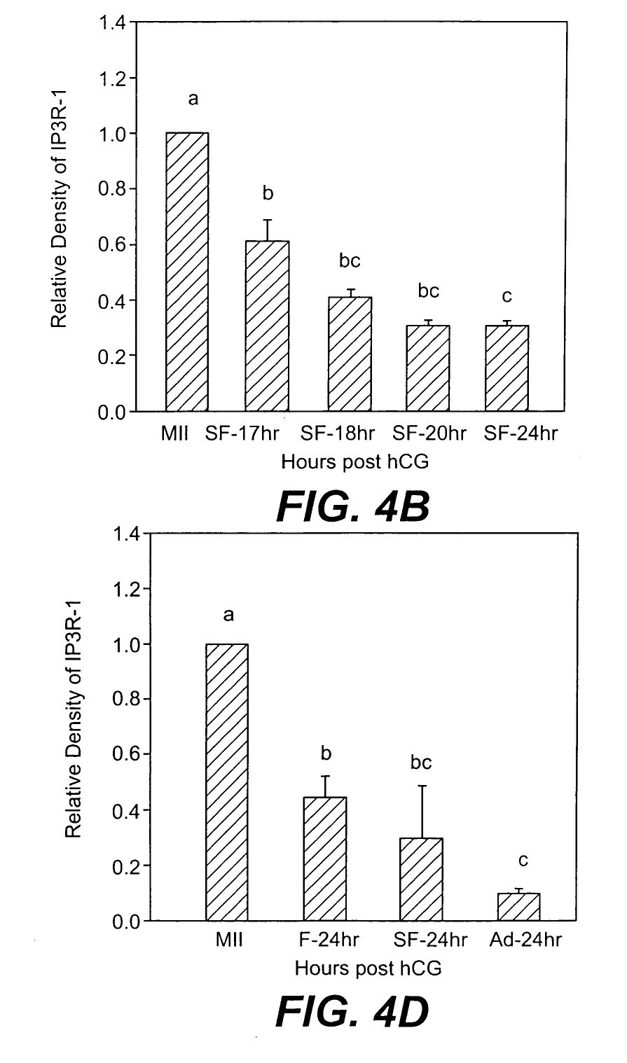

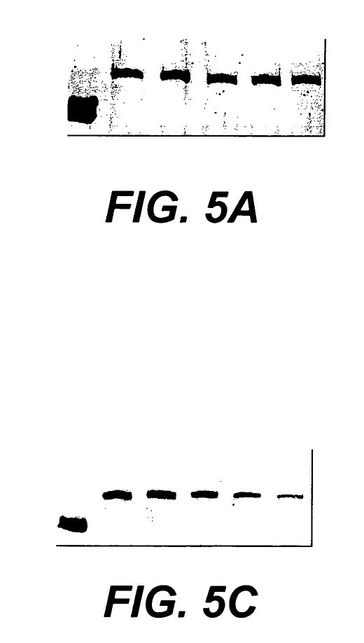

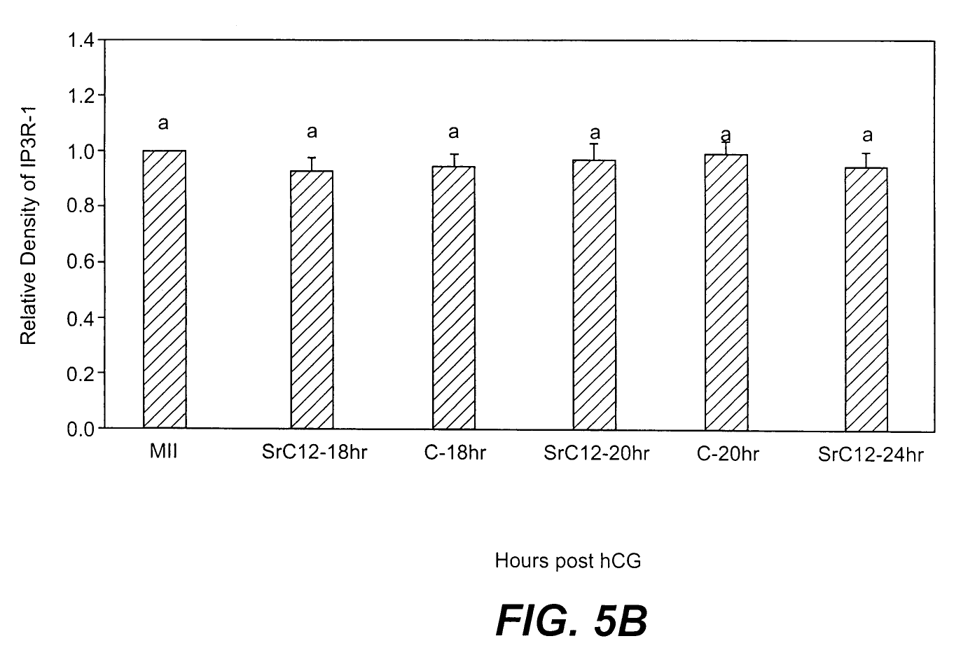

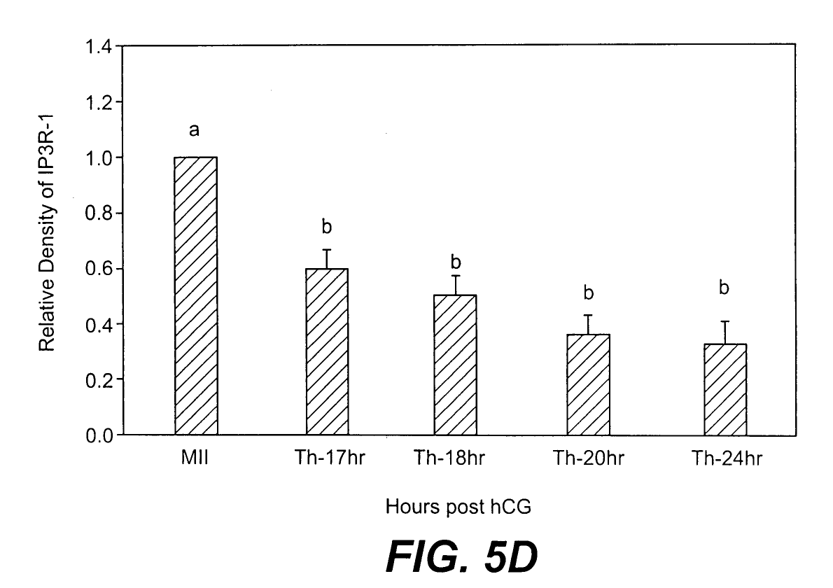

To test the notion that multiple [Ca²⁺]ᵢ rises are required for down-regulation of IP₃R-1, oscillations were initiated in mouse eggs by three different compounds that act on different molecular targets of the Ca²⁺ signaling pathway. Injection of SF, which triggers fertilization-like oscillations by presumably stimulating production of IP₃ (Jones et al., FEBS Lett. 437: 297-300 (1998)), induced marked down-regulation of the IP₃R-1 (FIG. 4A, B). The down-regulation of IP₃R-1 was persistent, although significant degradation was seen within 1 hr post-injection (FIG. 4A, B). Injection of adenophostin A, a non-hydrolyzable agonist of the IP₃R, also induced significant down-regulation of the receptor (FIG. 4C, D). Interestingly, this down-regulation was consistently greater than the degradation induced by fertilization (p<0.05) or by injection of SF (FIG. 4C, D). Finally, exposure of eggs to SrCl₂ for 2 or 4 hr failed to induce any changes in the amount of IP₃R-1 (FIG. 5 A, B). Together, these results suggest that down-regulation of IP₃R-1 during mouse egg fertilization is not exclusively due to the presence of multiple [Ca²⁺]ᵢ rises, but may be associated with [Ca²⁺]ᵢ oscillations initiated by activation of the phosphoinositide pathway.

Thimerosal Induces IP₃R-1 Down-regulation

Thimerosal, a thiol oxidizing agent, has been shown to induce [Ca²⁺]ᵢ rises without stimulating production of IP₃ (Hecker et al., Biochem. Biophys. Res. Comm. 159: 961-68 (1989); Bootman et al., J. Biol. Chem. 267: 25114-9 (1992); and Missiaen et al., J. Physiol. London 455: 623-40 (1992)). Thus, we investigated if oscillations initiated by co-incubation of eggs with this compound induced IP₃R-1 degradation. Thimerosal-mediated Ca²⁺ responses induced rapid and sustained down-regulation of the receptor (FIG. 5C, D). These data suggest that IP₃R-1 down-regulation in mouse eggs may not be exclusively signaled by activation of the phosphoinositidase pathway.

Patterns of [Ca²⁺]ᵢ Oscillations are Agonist-specific

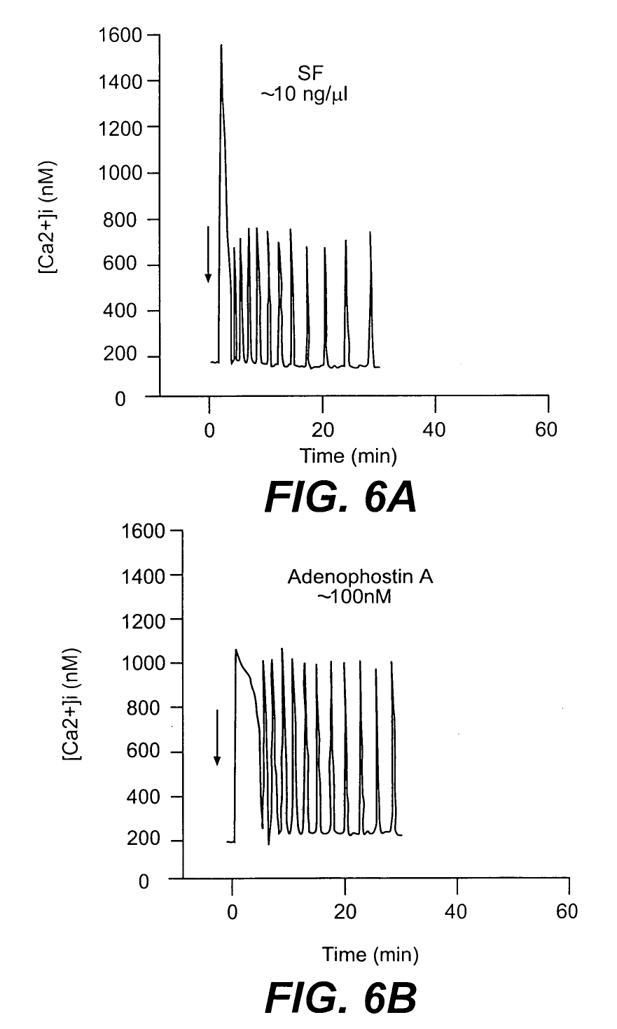

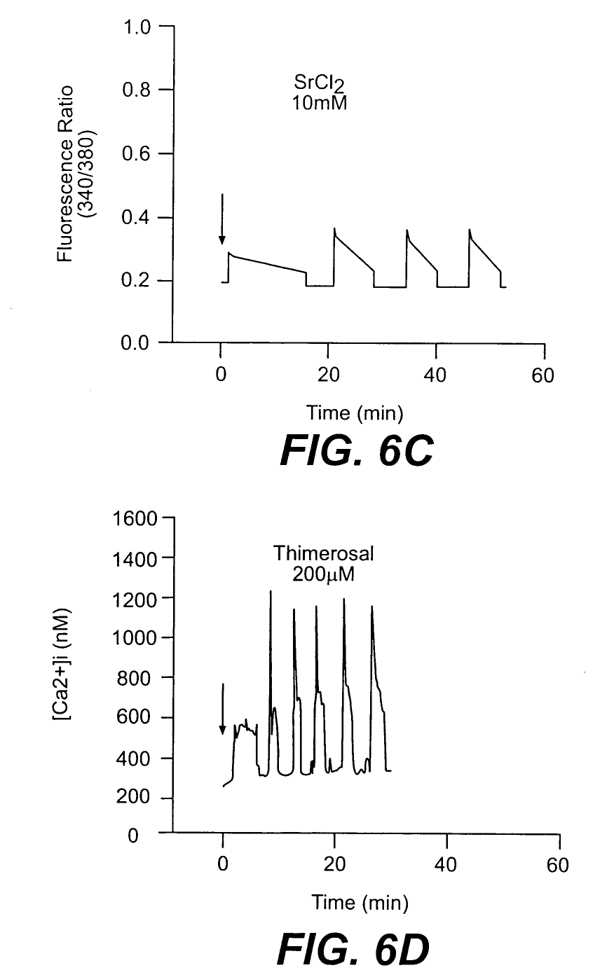

Due to the differential effects on EP₃R-1 down-regulation by the different agonists tested, we examined the Ca²⁺ responses triggered by each of these agonists. As expected, injection of SF (n=4 eggs) and adenophostin A (n=6 eggs) induced [Ca²⁺]ᵢ rises similar to those initiated by fertilization, but with higher frequency (p<0.05; FIG. 6A and B, respectively). On the contrary, eggs exposed to SrCl₂ (n=5 eggs) exhibited oscillations with lower frequency and these rises were different than those initiated by the other agonists in which the first rise and subsequent rises were very prolonged (Table 1; p<0.05; FIG. 6C). Eggs stimulated with thimerosal (n=9 eggs) showed Ca²⁺ responses with low frequency (p<0.05). However, the amplitude of thimerosal-induced spikes had comparable amplitude to those induced by SF and adenophostin A, although the first rise was of lower amplitude (Table 1; p<0.05). The amplitude of SrCl₂-induced rises was not compared to those induced by the other agonists, because fura-2 D, in this study, was calibrated to report intracellular Ca²⁺ levels and it is likely that the observed fluorescence changes represent changes in the concentrations of both cations (Hajnoczky et al., EMBO J. 16: 3533-43 (1997)). Despite the different Ca²⁺ profiles, the Ca²⁺ responses induced by all agonists (thimerosal excluded), appeared physiological as more than 90% of the eggs were activated and exhibited pronuclear formation (data not shown).

TABLE 1
Characteristics of [Ca²⁺]ᵢ rises induced by several common agonists in mouse eggs.

| [Ca²⁺]ᵢ | | | Amplitude | Amplitude | Duration | Duration |
|---|---|---|---|---|---|---|
| oscillation | # of | # of rises | of 1^{st} | of 3^{rd} | of 1^{st} | of 3^{rd} |
| inducing agonist | Eggs | in 5 min* | rise (nM) | rise (nM) | rise (sec) | rise (sec) |
| Adenophostin A | 6 | 3.9 ± 1.5^{a}*,* | 620 ± 30^{a, b} | 530 ± 100^{a} | 260 ± 65^{a} | 70 ± 22^{a} |
| SF | 4 | 5.0 ± 1.0^{a} | 870 ± 40^{a} | 440 ± 35^{a} | 330 ± 90^{a, b} | 60 ± 18^{a} |
| SrCl₂ | 5 | 1 ± 0.0^{b} | ND*** | ND | 740 ± 290^{b} | 360 ± 70^{b} |
| Thimerosal | 9 | 1 ± 0.4^{b} | 330 ± 30^{b} | 640 ± 90^{a} | 180 ± 44^{a} | 70 ± 16^{a} |

*The frequency of oscillations was monitored during a 5 min. period immediately after the return of the first rise to baseline values (Adenophostin A or SF) or after the addition of SrCl₂ or thimerosal. All types are means ± standard errors of the mean (SEM). The 3^{rd} rise was chosen arbitrarily to represent any subsequent spike in all treatments.
**Values that do not share a common superscript within columns are significantly different (p < 0.05).
***Not determined (“ND”), since Sr²⁺ may bind the fura-2 D and potentially interfere with the correct reporting of intracellular Ca²⁺ values.

Down-regulation of the IP₃R-1 is Mediated by the Proteasome

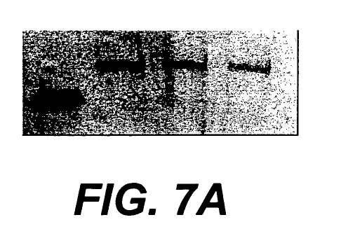

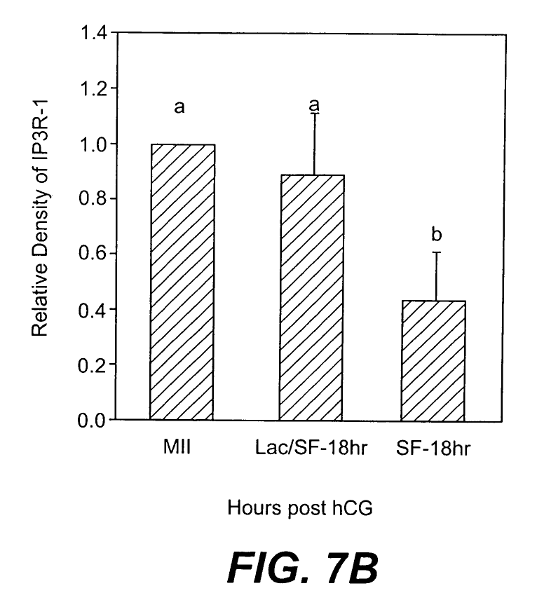

IP₃R-1 down-regulation in somatic cells has been shown to be mediated by the proteasome (Bokkala et al., J. Biol. Chem., 272: 12454-61 (1997); and Oberdorf et al., Biochem J. 339: 453-61 (1999)). The proteasome is also likely involved in down-regulation of specific proteins that allow fertilized mammalian eggs to exit the MII arrest (Kubiak et al., EMBO J. 12: 3773-8 (1993); for review see Whitaker, Rev. Reproduction 1: 127-135 (1996)). Thus, we determined if the degradation of IP₃R-1 induced by SF injection involved a similar pathway. To accomplish this, eggs were pre-incubated and injected with SF in the presence of lactacystin, a proteasome inhibitor. Activation was allowed to proceed for 2 hr, at which time the injected eggs were removed and prepared for Western blotting. The 2 hr time point was chosen because, by 1 hr post-injection, significant down-regulation of IP₃R-1 was already observed (FIG. 4A, B). Degradation of the receptor in SF-injected eggs incubated in lactacystin was markedly inhibited (FIG. 7). Furthermore, the effectiveness of the inhibitor on proteasome activity could also be deduced by the finding that cell cycle progression, as assessed by extrusion of the polar body, was clearly delayed in SF-injected eggs pretreated and incubated with the inhibitor (not shown).

Discussion

The results of this study in mouse eggs show a) that parthenogenetic activation induced by a single [Ca²⁺]ᵢ rise initiated by exposure to ethanol or ionomycin/DMAP does not induce down regulation of IP₃R-1; b) that initiation of oscillations by injection of SF, adenophostin A, or by exposure to thimerosal, evoked a marked decrease in the levels of IP₃R-1 similar to those observed during fertilization; c) that initiation of [Ca²⁺]ᵢ oscillations by exposure to SrCl₂ did not signal IP₃R-1 degradation; and d) that down-regulation of IP₃R-1 is likely to be mediated by the proteasome, since down-regulation was prevented by lactacystin, a proteasome inhibitor. Together, these data suggest that IPR3-1 down-regulation in mouse eggs after fertilization is associated with activation of the phosphoinositide/IP₃R system and that persistent IP₃ production, induced by the sperm during fertilization or by injection of SF in this study, may regulate the degradation of the IP₃R-1.

Mammalian oocytes and eggs closely control the number of IP₃R-1 before and after fertilization. During oocyte maturation, the increase and redistribution of the IP₃R-1 protein is intended to maximize the amount and spatial distribution of Ca²⁺ release following sperm penetration (Fujiwara et al., Dev. Biol. 156: 69-79 (1993); Mehlmann et al., Biol. Reprod. 51: 1088-98 (1994); and Shiraishi et al., Dev. Biol. 170: 594-606 (1995)). However, the role and regulation of the decline of IP₃R-1 numbers after fertilization is not fully elucidated, despite the fact that this decline may be specific since it is not observed in unfertilized aged eggs (Parrington et al., Dev. Biol. 203: 451-61 (1998) and present data) or in eggs activated by exposure to ethanol or ionomycin. These results indicate that IP₃R-1 down-regulation is not an effect of egg activation per se, but may be more closely associated with the number of [Ca²⁺]ᵢ rises or the mechanism by which the oscillations are initiated, both of which were tested in this study.

IP₃R-down-regulation studies in somatic cells have shown that degradation of the receptor requires persistent stimulation of PLC-coupled cell-surface receptors, since activation of these receptors that resulted in brief production of IP₃ was unable to induce receptor degradation (Oberdorf et al., Biochem J. 339-453-461 (1999)). To produce long-term stimulation in our study, mouse eggs were injected with SF. SF has previously been shown to induce prolonged [Ca²⁺]ᵢ oscillations that closely mimic those initiated by fertilization (Swann, Development 110: 1295-1302 (1990); Wu et al., Mol. Reprod. & Dev. 46: 176-89 (1997); and for review see Swann et al., BioEssays 19: 79-84 (1997)). These oscillations are mediated by the IP₃R as Ca²⁺ responses were suppressed by injection of the IP₃R-1 blocking monoclonal antibody 18A10 (Oda et al., Dev. Biol. 209: 172-85 (1999)). In the present study, SF induced a marked and persistent decline of IP₃R-1 similar to that observed following fertilization. SF has recently been shown to stimulate production of IP₃ in cell-free extracts from sea urchin eggs (Jones et al., FEBS Letts. 437: 297-300 (1998)), and thus, it is possible that it may induce IP₃R-1 down-regulation by stimulating long-term production of IP₃.

Our finding that injections of adenophostin A trigger down-regulation of IP₃R-1 supports this hypothesis. Adenophostin A, a product from Penicillium brevicompactum, is a full IP₃R agonist that is approximately 100-fold more potent than IP₃. Moreover, adenophostin A has greater affinity for the IP₃R and is not degraded by the IP₃ metabolizing enzymes (Takahashi et al., J. Biol. Chem. 269: 369-72 (1993)). These properties, which may allow this agonist to remain bound to the receptor for longer periods of time, may be responsible for the near total down-regulation of the IP₃R-1 observed in adenophostin A-injected eggs. The finding that thimerosal also induces down-regulation of the IP₃R-1 suggests that stimulation of the phosphoinositide pathway may not be the only mechanism to signal IP₃R-1 degradation in mammalian eggs. Thimerosal, an oxidizing agent that does not trigger IP₃ production, has been shown to increase the affinity of IP₃R-1 for 1P3 (Poitras et al., J. Biol. Chem. 268: 24078-82 (1993); Kaplin et al., J. Biol. Chem. 269: 28972-8 (1994); and Vanlingen et al., Cell Calcium 25: 107-14 (1999)) and it is possible that by this mechanism it may induce degradation of the IP₃R. Alternatively, thimerosal has been demonstrated to oxidize critical cysteine residues in the receptor inducing a change in the conformational state of the IP₃R (Sayers et al., Biochem. J. 289: 883-7 (1993)) and, in this manner, it may induce EP₃R-1 down-regulation. A conformational change has been shown to occur in the IP₃R following binding of 1P3, resulting in the opening of the channel (Mignery et al., EMBO J. 9: 3893-9 (1990)). This structural change may also be required for the degradation of the receptor (Zhu et al., J. Biol. Chem. 274: 3476-84 (1999)). Therefore, although thimerosal does not stimulate 1P3 production, it may signal IP₃R-1 degradation by inducing a similar modification of the receptor.

In contrast, SrCl₂ failed to induce down-regulation of IP₃R-1, despite inducing persistent oscillations. SrCl₂ has been suggested to induce [Ca²⁺]ᵢ oscillations by sensitizing a Ca²⁺-induced Ca²⁺ release (CICR) mechanism, although the precise mechanism is not known (Cheek et al., Development 119: 179-89 (1993)). The lack of effect of SrCl₂-induced responses on IP₃R-1 degradation is in marked contrast with the high rates of egg activation induced by this agonist. This demonstrates that SrCl₂-induced oscillations are capable of signaling the degradation of specific egg proteins, whose decline is known to be required to exit MII (Whitaker, Reviews in Reproduction 1: 127-35 (1996)). This clearly indicates that, in contrast to the situation of other egg proteins, the existence of [Ca²⁺]ᵢ oscillations and the resulting egg activation are not sufficient to induce down-regulation of IP₃R-1.

The conformational change induced by binding of IP₃ to its receptor has been suggested to signal IP₃R degradation by enhancing IP₃R ubiquitination and, consequently, signaling degradation by the proteasome (Bokkala et al., J. Biol. Chem. 272: 12454-61 (1997); and Oberdorf et al., Biochem. J. 339: 453-61 (1999)). In somatic cells, it has been shown that persistent stimulation of the phosphoinosftide pathway results in poly-ubiquitination of the IP₃R-1 (Bokkala et al., J. Biol. Chem. 272: 12454-61 (1997); and Oberdorfet al., (1999)), and studies using cells expressing mutant IP₃Rs-1, which were unable to bind IP₃, showed that these receptors were not degraded or ubiquitinated (Zhu et al., J. Biol. Chem. 274: 3476-84 (1999)). These studies also demonstrated that ubiquitinated EP₃Rs are degraded by the proteasome as addition of the cysteine protease and proteasome inhibitor, N-acetyl-Leu-Leu-norleucinal, and lactacystin, a highly specific inhibitor of the proteasome, both blocked the degradation of the receptor (Wojcikiewicz et al., J Biol. Chem. 271: 16652-5 (1996); (Bokkala et al., (1997); and Oberdorf et al., (1999)). Our results in mouse eggs showing that lactacystin blocked down-regulation of IP₃R-1 induced by injection of SF is evidence that the proteasome pathway is involved in the decline of IP₃R-1 numbers in eggs. Whether IP₃ binding to its receptor is the exclusive signal for degradation of the receptor in eggs is not known. Ca²⁺ or protein kinase C (PKC), both of which play a role in activation (Gallicano et al., BioEssays 19: 29-36 (1997)), may also participate in signaling IP₃R-1 degradation. Our findings that thimerosal, which does not stimulate the phosphoinositide pathway, triggers IP₃R degradation, and that SrCl₂, which induces oscillations without affecting receptor degradation, suggest that neither Ca²⁺ nor Ca²+-dependent PKC are critical or sufficient for IP₃R-1 demise in mouse eggs.

How the decline in IP₃R-1 numbers may affect the frequency and duration of [Ca²⁺]ᵢ oscillations remains to be determined. It is likely, however, that it may be involved in the cessation, or decline in frequency/amplitude, of fertilization/agonist-induced [Ca²⁺]ᵢ oscillations, which is observed as activated eggs progress to the pronuclear stage (Fissore et al., Dev. Biol. 166: 634-42 (1994); Jones et al., Development 121: 3259-66 (1995); and Parrington et al., Dev. Biol. 203: 451-61 (1998)). It is important to note that concomitant with these changes in IP₃R-1, two critical kinase activities also decline in eggs, those of maturation promoting factor and mitogen activated protein kinase (Moos et al., Biol. Reprod. 53: 692-9 (1995)). Thus, it will be necessary to determine whether one of these changes is more important than the other in the regulation/cessation of oscillations, or if both contribute equally. The use of eggs/zygotes with different numbers of IP₃R-1s but in similar cell-cycle stage/kinase levels, which can now be generated using the different agonists reported in this study, will allow us to discriminate the effect of IP₃R numbers and cell cycle stage on oscillations patterns in mammalian eggs.

In summary, the data presented here show that IP₃R-1 down-regulation in mouse eggs is induced by fertilization and by agonists that persistently stimulate the phosphoinositide pathway/IP₃R system. The data also show that the proteasome pathway is likely to mediate the degradation of the IP₃R-1.

### Example 3

### Method of Inducing Parthenogenetic Activation of an Oocvte Using Oscillogenin

Eggs are obtained from the oviducts of a CD-1 female mouse (6-12 weeks old) or other animal, superovulated by intraperitoneal injection of 5 IU of pregnant mare serum gonadotropin (PMSG; Sigma, St. Louis, Mo.) and is followed by 48 hr later injection of 5 I.U. of human chorionic gonadotropin (hCG; Sigma) to induce ovulation. Eggs are recovered 14 h post-hCG into a Hepes-buffered solution (TL-Hepes; Parrish et al., Biol. Reprod. 38: 1171-80 (1988)) supplemented with 10% heat-treated calf serum (CS; Gibco, Grand Island, N.Y.). Cumulus cells are removed with bovine testes hyaluronidase (Sigma).

Microinjection procedures are used as described in Wu et al., Mol. Reprod. Dev. 46: 176-89 (1997) and Wu et al., Mol. Reprod. Dev. 49: 37-47 (1998). In brief, eggs are microinjected using Narishige manipulators (Medical Systems Corp.; Great Neck, N.Y.) mounted on a Nikon Diphot microscope (Nikon, Inc., Garden City, N.Y.). Glass micropipets are filled by suction of a microdrop containing 0.5 mM fura 2 dextran (fura 2D, dextran 10 kDa, Molecular Probes; Eugene, Oreg.) or sperm extract (1-20 mg/ml protein concentration). Solutions are expelled into the cytoplasm of eggs by pneumatic pressure (PLI-100, picoinjector; Medical Systems Corp., NY). The injection volume is about 5 to about 10 pl and results in final intracellular concentration of the injected compounds of approximately 1% of the concentration in the injection pipette. Injections of sperm factor (SF) results in a Ca²⁺ oscillation and complete activation of oocyte development.

Fura 2D fluorescence is monitored as previously described by Wu et al., (1997 and 1998 above), which monitors Ca²⁺ oscillations. Briefly, excitation wavelengths are at 340 and 380 nm and the emitted light is quantified, after passing through a 500-mn barrier filter by a photomultiplier tube. The intensity of excitation light is attenuated by neutral density filters, and the fluorescent signal is averaged for the whole egg. [Ca²+]ᵢ concentrations (Rₘᵢₙ and Rₘₐₓ) are calculated according to Grynkiewickz et al., J. Biol. Chem. 260: 3440-50 (1985); Poenie, Cell Calcium 11:85-91 (1990); Fissore et al., Dev. Biol. 159: 122-30 (1993) and Wu et al., (1997 and 1998). Determining parthenogenetic activation can be performed by visualization of whether the cell forms a pronucleus or undergoes a first cleavage event. Parthenogenic activation can also be assessed biochemically by assessing whether histone HI is down-regulated, DNA synthesis is up-regulated, or by other methods which would be known to the skilled artisan.

[Ca²⁺]ᵢ monitoring for determining parthenogenetic activation of the mouse eggs starts 30-45 min. after injection of fura 2D, which is approximately 15 hr post-hCG administration. Eggs are monitored individually in 50 μl medium placed on a glass coverslip on the bottom of a culture dish covered with paraffin oil. Fluorescence ratios are obtained every 4 sec for 15 to 30 min. Prior to the injection of oscillogenin, fluorescent recordings are taken to establish baseline values. Readings are taken for 1 sec at each wavelength. [Ca²⁺]ᵢ monitoring is completed before eggs have reached 22 h post-hCG. All sperm extracted fractions are tested at 1 mg/ml protein concentration.

### Example 4

### An Anti-oscillogenin Antibody

Antibodies can also be prepared by subcloning the oscillogenin cDNA into a glutathione S-transferase (GST)-gene based expression vector pGEX 3 system. The correct orientation and position of the oscillogenin insert is confirmed by sequencing of nucleotides in the site of transcription initiation. The construct is then transformed into Escherichia coli BL21 strain, and GST-oscillogenin fusion protein expression is stimulated by addition of IPTG. The expressed GST fusion protein is purified by affinity chromatography and separated from its GST fusion partner by cleavage with the protease Factor Xa (Pharmacia). Then, the Factor Xa is removed from the preparation by benzarnidine Sepharose 6B beads. Protein purity before injection into rabbits is checked using SDS-PAGE and Coomassie blue staining.

Purified recombinant or extracted oscillogenin is injected into rabbits to produce polyclonal antibodies. The immunization procedure involves an initial injection (40 μg of oscillogenin) followed by two boost injections of 20 μg of protein 3 to 4 weeks apart.

Polyclonal antibodies thus raised can be affinity purified, eluted via a pH gradient, and stored in a borate buffer.

Although the present invention has been described in detail with reference to examples above, it is understood that various modifications can be made without departing from the spirit of the invention. All cited patents and publications referred to in this application are herein incorporated by reference in their entirety.

1. A method of isolating an oscillogenin from sperm comprising:
(A) preparing a sperm cytoplasmic fraction;
(B) isolating oscillogenin by sequentially processing the sperm cytoplasmic fraction through a HiTrap blue affinity FPLC chromatographic column, a hydroxyapatite FPLC column, and a Superose 12 FPLC chromatographic column; and
(C) obtaining a fraction with [Ca²⁺]ᵢ releasing activity.

2. A method of enhancing oocyte activation comprising the step of:
(a) introducing oscillogenin into an oocyte prior to, simultaneously with, or immediately after injecting or fusing the oocyte with a sperm or other cell nuclei, wherein said oocyte has been treated, before or after oscillogenin injection, to remove or inactivate its endogenous nucleus.

3. The method of claim 2, wherein the oocyte is a mammalian oocyte.

4. The method of claim 3, wherein the mammalian oocyte is a human oocyte.

5. The method of claim 2 further comprising incubation of the injected oocyte in a medium containing Ca²⁺.

6. The method of claim 2, wherein the sperm is a mammalian sperm.

7. The method of claim 6, wherein the mammalian sperm is selected from the group consisting of: primate, bovine, porcine, ovine, equine, feline, canine, murine and caprine.

8. The method of claim 2, further comprising the step of injecting the oocyte with at least one agent which additionally enhances divalent cation release or a combination of such agents.

9. The method of claim 8, wherein the agent is selected from the group consisting of: a calcium ionophore, a protein kinase inhibitor and a phosphatase.

10. The method of claim 9, wherein the calcium ionophore is selected from the group consisting of: ionomycin and A23187.

11. The method of claim 9, wherein the protein kinase inhibitor is selected from the group consisting of: 6-dimethylaminopurine (DMAP), staurosporine, butyrolactone, roscovitine, p34(cdc2) inhibitors, 2-aminopurine and sphingosine.

12. The method of claim 9, wherein the phosphatase is select from the group consisting of:. phosphatase 2A and phosphatase 2B.

13. The method of claim 2, which further comprises allowing said activated oocyte to. develop into an embryo.

14. The method of claim 13, wherein said embryo is non-human, and is implanted into a female surrogate.

15. The method of claim 14, wherein said implanted embryo is allowed to develop into a viable, non-human offspring.

16. The method of claim 2, wherein said activated oocyte is cultured to produce a blastocyst.

17. The method of claim 16, which further comprises culturing all or part of the inner cell mass of said blastocyst on a feeder layer to produce a cultured inner cell mass.

18. The method of claim 17, wherein said cultured inner cell mass is transferred onto a different feeder layer in order to prevent differentiation of said cultured inner cell mass.

19. The method of claim 18, wherein said cultured inner cell mass is cultured to produce a cultured inner mass cell line.

20. A method of enhancing intracytoplasmic sperm injection (ICSI) comprising the step of injecting an oocyte with oscillogenin either before or after a sperm or sperm nucleus is inserted into the oocyte.

21. The method of claim 20 further comprising incubation of the injected oocyte in a medium containing Ca²⁺.

22. The method of claim 20, wherein the oocyte and sperm are mammalian.

23. The method of claim 22, wherein the oocyte is selected from the group consisting of: primate, bovine, porcine, ovine, equine, feline, canine, murine and caprine.

24. The method of claim 22, wherein the sperm is selected from the group consisting of: primate, bovine, porcine, ovine, equine, feline, canine, murine and caprine.

25. The method of claim 20, further comprising the step of injecting the oocyte with at least one agent which enhances divalent cation release.

26. The method of claim 25, wherein the agent is selected from the group consisting of: a calcium ionophore, a protein kinase inhibitor and a phosphatase.

27. The method of claim 26, wherein the calcium ionophore is selected from the group consisting of: ionomycin and A23187.

28. The method of claim 26, wherein the protein kinase inhibitor is selected from the group consisting of: 6-dimethylaminopurine (DMAP), staurosporine, butyrolactone, roscovitine, p34(cdc2) inhibitors, 2-aminopurine and sphingosine.

29. The method of claim 26, wherein the phosphatase is select from the group consisting of: phosphatase 2A and phosphatase 2B.

30. A method of parthenogenically activating an oocyte comprising the step of injecting oscillogenin into the oocyte.

31. The method of claim 30, wherein the oocyte is a mammalian oocyte.

32. The method of claim 31, wherein the mammalian oocyte is selected from the group of mammals consisting of: human, primate, bovine, porcine, ovine, equine, feline, canine, murine and caprine.

33. A method of predicting sperm [Ca²⁺]ᵢ releasing activity comprising measuring oscillogenin concentration in a sperm sample.

34. A kit for predicting sperm [Ca²⁺]ᵢ releasing activity comprising a labeled agent which recognizes and binds to oscillogenin or a nucleic acid encoding oscillogenin.

35. The kit of claim 34, wherein the agent is an anti-oscillogenin antibody.

36. The kit of claim 34, wherein the agent is a nucleic acid probe which binds to oscillogenin mRNA.

37. A nucleic acid encoding an oscillogenin.

38. (canceled)

39. A vector comprising the nucleic acid of claim 37.

40. The nucleic acid of claim 37, wherein the oscillogenin is a mammalian oscillogenin.

41. The nucleic acid of claim 40, wherein the mammalian oscillogenin is selected from the listing consisting of human, bovine, porcine, ovine, equine, feline, canine, murine and caprine.

42. An oscillogenin protein encoded by the nucleic acid of claim 37.

43. (canceled)

44. (canceled)

45. An isolated oscillogenin obtained by the method of claim 1.

46. A recombinant oscillogenin protein obtained by:
(A) inserting the vector of claim 39 into a suitable host;
(B) incubating said host under suitable conditions to produce oscillogenin; and
(C) isolating oscillogenin protein from said host.

47. A composition for activating oocytes comprising an oscillogenin protein and a pharmaceutically acceptable carrier.

48. The composition of claim 47 further comprising at least a phosphatase, a calcium ionophore or a protein kinase inhibitor.

49. An antibody or immunogenic fragment thereof which recognizes and binds to oscillogenin.

50. The antibody of claim 49, wherein the antibody is a monoclonal antibody.

51. The antibody or immunogenic fragment of claim 49, wherein the immunogenic fragment is selected from the group consisting of: Fab, scFv, F(ab′)2 and Fab′.

52. The antibody or immunogenic fragment of claim 49, wherein the antibody or immunogenic fragment is a labeled antibody.

53. The antibody or immunogenic fragment of claim 52, wherein the antibody or immunogenic fragment is labeled with an isotope or a fluorescent label.

54. The antibody of claim 53, wherein the fluorescent label is rhodamine, fluorescein or Rhodamine GreenO.

55. A method for inhibiting sperm fertility comprising the step of administering an agent which inhibits oscillogenin activity in sperm
